(19) United States
(12) Patent Application Publication
(10) Pub. No.: US 2016/0273010 A1
(43) Pub. Date: Sep. 22, 2016

(54) MELT COMPOUNDING AND FRACTIONATION OF LIGNOCELLULOSIC BIOMASS AND PRODUCTS PRODUCED THEREFROM

(71) Applicants: Scott RENNECKAR, Vancouver (CA); Justin BARONE, Roanoke, VA (US); Young KIM, Blacksburg, VA (US)

(72) Inventors: Scott RENNECKAR, Vancouver (CA); Justin BARONE, Roanoke, VA (US); Young KIM, Blacksburg, VA (US)

(73) Assignee: Virginia Tech Intellectual Properties, Inc., Blacksburg, VA (US)

(21) Appl. No.: 15/033,833

(22) PCT Filed: Oct. 31, 2014
(86) PCT No.: PCT/US2014/063480

§ 371 Date: May 2, 2016

Related U.S. Application Data
(60) Provisional application No. 61/931,908, filed on Jan. 27, 2014.
(60) Provisional application No. 61/897,975, filed on Oct. 31, 2013.

(51) Int. Cl. C12P 19/14; C08H 8/00; C13K 1/02; C12P 19/02

(52) U.S. Cl. CPC C12P 19/14; C12P 19/02; C08H 8/00; C13K 1/02; C12P 2201/00

(57) ABSTRACT

Methods and processes to fractionate and/or convert cellulosic material into accessible sugar and chemical intermediates is provided. A method of embodiments of the invention includes processing lignocellulosic biomass by mixing lignocellulosic biomass and glycerol to form a biomass slurry, and heating and shearing the biomass slurry at a temperature ranging from 100° C. to 300° C. for an amount of time to disrupt inter- or intra-polymer linkages of the biomass. The demonstrated swelling and maceration of a biomass material in the presence of a solvent at elevated temperatures and under shearing, provides a processing window to efficiently extract lignin and convert cellulosic material into useful sugars at high conversion rates.

### CROSS-REFERENCE TO RELATED APPLICATIONS

The present application relies on the disclosure of and claims priority to and the benefit of the filing dates of U.S. Provisional Application No. 61/897,975, filed Oct. 31, 2013, and U.S. Provisional Application No. 61/931,908, filed Jan. 27, 2014, the disclosures of which are hereby incorporated by reference herein in their entirety.

### FIELD OF THE INVENTION

The present invention relates to pretreatment processes for fractionating lignocellulosic materials and methods for enhancing saccharification of fermentable sugars.

### BACKGROUND

Current commercial energy sources have inherent problems with regard to sustainability because of their reliance on fossil fuels. Based on the conservation of matter, burning fossil fuels adds carbon stored from previous millennia to the earth's atmosphere. At a consumption rate of 85 million barrels of oil a day, the earth does not have the resources to meet this demand from stored fossil fuels nor the environmental capacity to handle the released carbon. (Kerr, R. A., World oil crunch looming? Science (Washington, D.C., U. S.), 2008. 322 (5905): p. 1178-1179). However, it is not only fuel that is at stake. In fact, 84% of a barrel of oil goes towards low-value fuel while 16% goes toward much higher value chemicals (roughly 13 million barrels/day). (US-Energy-Information-Administration. Oil (petroleum). 2012 Jan. 31, 2012; http://www.eia.gov/kids/energy.cfm?page=oil_home-basics).

Chemicals provide us with everything from pharmaceuticals to furniture, and chemicals cannot currently be derived from energy sources like solar or wind. Unlike automobiles, which have the potential to be powered by renewable energy sources independent of fossil fuels and derived from cleaner sources, once fossil fuels are no longer sustainable economically and environmentally, chemicals, will need to be obtained from another source.

Currently, easily accessed sugars from pressed sugarcane or hot steeped corn have supplied the Brazilian and U.S. ethanol biorefineries on an industrial scale with the former demonstrating the possibilities of an advanced bioeconomy. This success is predicated on the conversion of sucrose to ethanol. Starch, an energy reserve polymer of glucose, is easily hydrolyzable and has been used as the low hanging fruit to meet the blending requirements of the Renewable Fuels Standard (RFS) set out within the Energy Independence and Security Act of 2007. (110th-Congress, Energy Independence and Security Act of 2007. (2007)). U.S. farmers were responsive to market demands when addressing the corn ethanol policy mandate in the RFS because they did not need to make significant changes to their farming practices. Furthermore, advanced technology breakthroughs were not required to take corn starch through a conversion process because it is a familiar process used by grain distilleries. The market demand for corn ethanol, and the economic success of farmers from selling corn at record bushel prices, has allowed production to approach the “blend wall” for the E10 fuels. (Service, R. F., Is there a road ahead for cellulosic ethanol? Science (Washington, D.C., U. S.), 2010. 329 (5993): p. 784-785). This limit is where production meets the mandated demand of approximately 14 billion gallons.

Corn-derived ethanol, however, has come under scrutiny for its intensive farming practices (e.g., water, fertilizer, and pesticide loading requirements), increasing food prices as ethanol production has increased, the fossil fuel requirements for ethanol production and total CO₂ output throughout its lifecycle, and the future sustainability of production without subsidies for blenders. (Schwietzke, S., W. M. Griffin, and H. S. Matthews, Relevance of Emissions Timing in Biofuel Greenhouse Gases and Climate Impacts. Environ. Sci. Technol., 2011. 45 (19): p. 8197-8203). Due to these challenges, cellulosic derived energy alternatives to corn-derived ethanol are being explored.

Cellulose is produced globally at 100 billion tons, greatly surpassing the amount of available glucose from food and agricultural crop based sources like sugar beets. (U.S. Department of Energy, (2011) U.S. Billion-Ton Update: Biomass Supply for a Bioenergy and Bioproducts Industry. R. D. Perlack and B. J. Stokes (Leads), ORNL/TM-2011/224. Oak Ridge National Laboratory, Oak Ridge, Tenn. http://bioenergykdf.net/, p. 227). Use of cellulose as an energy source, however, is not without its complications.

Technological barriers exist in accessing the fermentable sugars locked within the cellulose. The majority of plant biomass consists of at least 2/3 polysaccharides that are structural polymers within the cell wall. Three of the main hurdles for accessing, and then liberating, these polysaccharides, and subsequently sugars, from abundant cellulosic sources include:

1) the recalcitrance of cellulose due to the multiscale hierarchical structure of cellulose microfibrils with inaccessible cellulose cores. All native celluloses occur as supramolecular structures in microfibril aggregates of cellulose chains. The packing is highly efficient where intermolecular hydrogen bonds between cellulose chains provide for high sheet stability and van der Waals bonds hold a stack of sheets together forming a microfibril aggregate.

2) the presence of lignin, a poly-aromatic material that shields the polysaccharide backbone. Lignin increases the cost of liberating the polysaccharide components (e.g., cellulose) from biomass (cellulose is only one component of biomass, which is sold on a dry biomass basis, and not a dry cellulose basis).

3) hemicellulose components that are labile and have the potential to form inhibitive fermentation products during standard acidic pretreatment or steam-explosion processes.

Overcoming these hurdles is critical and advanced biofuels and platform chemicals derived from plants are currently dependent upon the availability of sugars within plant biomass to be transformed by one of the many routes: chemically via catalytic reduction to alkanes; microbiotically into alcohols and/or acids; or heterotrophically via algae to oil.

Indeed, advancements in the pretreatment processes for biomass intended for conversion to fermentable sugars have shown improvement in hydrolysis at different scales, however, these systems have not widely been viewed as a final solution for the pretreatment of biomass. The problem is that current technologies have failed to attract cellulosic conversion development at the commercial scale and the current treatment technologies are based on corrosive compounds (acids and bases) that limit processing equipment choices (Kamm, B., P. R. Gruber, M. Kamm (eds.), Biorefineries—Industrial Processes and Products: Status Quo and Future Directions. 2010, ISBN: 3527329536, p. 949).

Acid treatment, such as dilute acid hydrolysis, is a well-known method for breaking lignin-carbohydrate linkages, hydrolyzing the hemicellulose components, and providing access to the cellulose microfibrils via disruption of the cell wall organization. Effective as these treatments may be, however, these processes are harsh and can have negative impacts on the environment. Acid treatments, in particular, are corrosive on processing equipment. Additionally, the lignin properties decline and become crosslinked (Bozell), and residual sulfate ester groups on cellulose can have an inhibitory effect on enzyme saccharification (Roman). Accordingly, and in spite of acid pretreatment processes being continually studied, significant drawbacks exist to acid-pretreatment processes.

Alternatively, alkaline treatments have been explored as a method for liberating cellulose from lignin-carbohydrate linkages. Common alkaline treatments, such as ammonium or calcium hydroxide treatments, can cause the biomass to swell providing a plasticizing effect on the biomass material which disrupts some of the organization of the cell wall make-up. As with acid treatments, however, alkaline treatments are not ideal and present myriad challenges as well. Alkaline based systems often require specialized equipment (ammonia fiber expansion) and/or are time intensive (soak for hours for ammonia percolation). Furthermore, before the enzymatic hydrolysis steps can be performed, wherein the biomass is converted into fermentable sugars, a significant amount of acid is necessary for neutralizing the basic pH of alkaline-based cellulosic material pretreatment systems.

Other techniques, such as organosolv pulping of biomass, dissolution of biomass in ionic liquids, or a cellulose solvent and organic solvent lignocellulose fractionation method (COSLIF) (e.g., cellulose dissolution in phosphoric acid), have also been explored for the pretreatment of biomass. These techniques are capable of providing high conversion factors because they address commercial and production issues associated with lignin hindering access to the polysaccharide surface, disrupt the crystalline structure of cellulose, and lower the pretreatment reaction temperatures to reduce problems with by-product formation. (Tadesse, H. and R. Luque, Advances on biomass pretreatment using ionic liquids: an overview. Energy Environ. Sci., 2011. 4 (10): p. 3913-3929). Notwithstanding the benefits of these newer methods, these techniques have their shortcomings as well.

There are myriad complications that surround the use of ionic liquids, such as their efficacy in the presence of impurities, the full recovery of the liquids from the residual biomass, difficulty isolating dissolved lignin, and potential cost. For instance, processes for recycling a solvent to remove yield reducing compounds (including water) or processes engineered for multiple washings of the biomass are typically necessary to effectively remove all of the solvent from a biomass. Failure to remove these solvents can have adverse impacts on saccharification and/or fermentation—both of which ultimately drive up production costs. The efficiency of the solvent based approach is proportional to the energy required to remove impurities which negatively effects biomass conversion. As such, the use of green solvents remains difficult to scale for industrial application.

A need exists for solutions which will mitigate the industrial reliance on fossil fuels. With the global population at 7 billion people and growing, it is critical that commercially viable cellulosic conversion technologies are developed to fractionate an abundant stream of accessible sugars that can be made useful for further downstream conversion and processing.

### SUMMARY

The first step in the digestion of cellulosic material according to the methods disclosed is pretreatment of the lignocellulosic biomass. In particular, pretreatments utilizing thermal processing (i.e., melt compounding equipment) to shear biomass at elevated temperatures in the presence of the benign solvent glycerol. It has been noted that ethylene glycol and glycerol can be used to protect thermally sensitive biopolymers like keratin or starch because it can prevent dehydration and other mechanisms leading to polymer degradation. Additionally, it has been reported that glycerol can plasticize biomass when heated above the glass transition (Frazier and others) and that glycerol-plasticized wood undergoes irreversible change in swelling when heated beyond the glass transition (T_{g}) (Chowdhury/Frazier).

Solvolysis was implicated in the mechanism of this change resulting in a reduction of an effective crosslink density. Additional rheological analysis demonstrated that glycerol plasticized wood's polymeric organization is highly fragile, i.e., undergoes extreme conformational changes during its T_{g}. Without being bound by theory, rheological work shed light into two possible mechanisms involved in this novel pretreatment process.

The first is that the plant cell wall has undergone significant plasticization and reorganization with key linkages connecting the cell wall polymers breaking after heating and shearing the samples in glycerol under a broad range of pressure and temperature. This change impacts nature's protective cell wall polymer network, disrupting it enough to provide access to the cellulosic material for hydrolytic enzymes. The second is the irreversible swelling arising from a change in the effective crosslink density of the amorphous phase resulting in a higher specific surface area substrate.

Processes known in the art have shown that glycols (usually with catalysts or long treatment times) can be used as a biomass pretreatment step; however, the inventors have discovered a technological breakthrough using melt processing equipment in the presence of a solvent, such as, glycerol. The combination of high throughput, high solids loading, and enhanced hydrolysis conversion demonstrates that the disclosed pretreatment process is an improved pretreatment process for generating streams of hydrolyzable sugars or a purified cellulose and solvent extractable lignin as a way to create materials for a bioeconomy.

Described herein are methods and processes to fractionate and convert stored solar energy, in the form of cellulosic material, into accessible sugar and chemical intermediates.

As will be described, the demonstrated swelling and maceration of the cellulosic material (e.g., cellulosic material, lignocellulosic material, lignocellulosic biomass, or “biomass”) in the presence of at least one solvent (e.g., at least one polyhydric alcohol such as glycerol), at elevated temperature, provides a processing window that offered enough bond breakage to make extraction of lignin efficient and the biomass swollen enough to allow for over 80% or even 90% conversion into glucose. The lignocellulose complex of biomass contains four main types of bonds that provide linkages within the individual components of lignocellulose (intrapolymer linkages) and that connect the individual components together to form the complex (intrapolymer linkages), i.e., ether, ester, and hydrogen bonds, as well as carbon-to-carbon bonds. See Harmsen, Huijgen, Lopez, and Bakker, Literature Review of Physical and Chemical Pretreatment Processes for Lignocellulosic Biomass, Food and Biobased Research, Wageningen University and Research Center, ECN-E-10-013 (September 2010).

Glycerol, also known as glycerine, is a non-toxic and benign solvent that is currently a by-product of the 2 billion gallon capacity biodiesel industry (EIA). Moreover, glycerol is a solvent capable of plasticizing macromolecules such as cellulose, and hemicelluloses. Currently, glycerol is used to plasticize proteinaceous biopolymers, like keratin, and starch materials during melt processing, and recently has been shown to be capable of lowering the glass transition of wood by 80° C.

The inventors have shown that using glycerol in processing biomass, in particular a thermolytic heat pretreatment processes such as holt-melt extrusion or melt compounding, is superior to current solvents used in pretreatment processes. Unlike conventional solvents, glycerol has a high boiling point and is capable of interacting with the highly functional biopolymers through secondary interactions such as hydrogen bonding. Without being bound by theory, is believed the use of glycerol in thermolytic pretreatment processes will offer at least the following advantages to current industrial pretreatment practices:

(1) glycerol is capable of swelling cellulosic materials including other biobased macromolecules and enhancing enzyme access on polysaccharides during downstream processing when cellulosic materials are subject to thermolytic pretreatment processes;

(2) glycerol is capable of protecting one or more polysaccharide components against dehydration and degradation when lignocellulosic materials are subject to thermolytic pretreatment processes;

(3) glycerol is capable of protecting polyphenolic such as lignin and other minor components such as phytochemical components against acid-catalyzed condensation and oxidation when cellulosic materials are subject to thermolytic pretreatment processes;

(4) glycerol is capable of limiting inhibitive fermentation products when cellulosic materials are subject to thermolytic pretreatment processes;

(5) glycerol is able to protect xylan from depolymerization at typical thermolytic processing temperatures; and

(6) a readily bleachable pulp is created for obtaining a cellulose material having high alpha-cellulose content.

Material resulting from thermolytic pretreatment processes performed in the presence of at least one cellulosic solvent (e.g., a polyhydric alcohol such as glycerol) can be converted in high yield into simple fermentable sugars, while maintaining a high molecular weight, non-condensed lignin, which can be recovered in good yield. The inventors believe that the controlled degradation of cellulosic material, in a stable environment, can be exploited as the initial pretreatment step for fractionation to separate out the polysaccharide components from lignin.

Considering challenges associated with the scalability of current processes, the inventors have developed a biomass treatment process using existing industrial polymeric compounding equipment to continuously process biomass in the presence of at least one cellulosic solvent, without additional corrosive processing aids. It is believed that these green processes can increase the environmental and social values of final products in terms of sustainability, while reducing the use of strong chemicals and increasing the use of different types of biomass including biomass waste from agricultural industries.

Provided is a method of processing lignocellulosic biomass, comprising:

a. mixing lignocellulosic biomass and glycerol to form a biomass slurry;

b. and heating and shearing the biomass slurry at a temperature ranging from 100° C. to 300° C. under the broad range of pressure during the reaction for an amount of time to disrupt inter- and/or intra-polymer linkages of the biomass. Such methods can further comprise fractionating cellulose, hemicellulose, and/or lignin from the biomass slurry, and/or converting one or more fractions to sugars.

For example, a method of processing lignocellulosic biomass is provided, the method comprising: providing lignocellulosic biomass and at least one solvent; providing and heating a mixer to a temperature of between 100° C. to 300° C.; adding the biomass and the solvent to the mixer; mixing the biomass and solvent into a biomass slurry; and melt compounding the biomass slurry under shearing and heating for an amount of time to cause disruption of inter- or intra-polymer linkages of the biomass.

In an embodiment, the methods described herein disclose a process for pretreating a cellulosic material for hydrolysis, comprising:

a. mixing at least one cellulosic material and at least one cellulosic solvent in a reactor to form a biomass slurry; and

b. heating the biomass slurry at a temperature in the range from equal to or more than 100° C. to equal to or less than 300° C. to obtain a pretreated cellulosic material for hydrolysis.

In yet another embodiment, the methods described herein disclose a process for hydrolyzing a cellulosic material comprising:

a. mixing at least one cellulosic material and at least one cellulosic solvent in a reactor to form a biomass slurry;

b. heating the biomass slurry at a temperature in the range from equal to or more than 100° C. to equal to or less than 300° C. to obtain a pretreated cellulosic material; and

c. hydrolyzing the pretreated cellulosic material with at least one enzyme that can hydrolyze the pretreated cellulosic material into at least one fermentable sugar.

In still yet another embodiment, the methods described herein disclose a process for isolating lignin from cellulosic material for hydrolysis, comprising:

a. mixing at least one cellulosic material and at least one cellulosic solvent in a reactor to form a biomass slurry; and

b. heating the biomass slurry at a temperature in the range from equal to or more than 100° C. to equal to or less than 300° C. to obtain a pretreated cellulosic material; and

c. extracting lignin from the pretreated cellulosic material, wherein the extracted lignin has a number average molar mass (Mₙ) in the range from equal to or more than 1,000 to equal to or less than 10,000.

### BRIEF DESCRIPTION OF THE DRAWINGS

FIG. 1 is a graph illustrating the total glucan digestibility of sweet gum samples pretreated according to the methods described herein.

FIG. 2 is a graph illustrating the effect of glycerol on the total glucan digestibility of sweet gum samples pretreated as described herein.

FIG. 3 is a graph illustrating the effect of particle size on the total glucan digestibility of sweet gum samples pretreated as described herein.

FIG. 4 is a graph illustrating the total glucan digestibility of corn stover samples pretreated according to the methods described herein.

FIG. 5 is an image of an infrared spectrum of the lignin isolated according to the methods described herein.

FIG. 6 is a graph of the functional group content of the lignin isolated according to the methods described herein.

FIG. 7 is a graph of the functional group content of the lignin isolated according to the methods described herein.

FIG. 8 is a graph of the carboxyl group content of the lignin isolated according to the methods described herein.

FIG. 9 is an image of a Gas Phase Chromatography (GPC) trace of the lignin isolated according to the methods described herein.

FIG. 10 is a process flow diagram (PFD) representing an overview of the method used for processing BSG as described herein.

FIG. 11 is a process flow diagram (PFD) representing an overview of the washing methods used for processing BSG as described herein.

FIG. 12A is a process flow diagram (PFD) illustrating the mass balance of BSG for the water washing and drying steps for BSG as described herein.

FIG. 12B is a process flow diagram (PFD) illustrating an extraction procedure for BSG using an enzymatic detergent as described herein.

FIG. 13 is a process flow diagram (PFD) illustrating the mass balance of BSG for without the enzyme extraction and glycerol washing steps for BSG as described herein.

FIG. 14 is an image of TMR pulp prepared as described herein.

### DETAILED DESCRIPTION OF THE EMBODIMENTS

### Definitions

The singular forms “a”, “an” and “the”, as used herein, mean to include the plural forms as well, unless the context clearly indicates otherwise.

The terms “about” or “approximately” as used herein may be used interchangeably and when used in conjunction with a stated numerical value or range denotes somewhat more or somewhat less than the stated value or range, to within a range of ±10% of that stated.

Cellulolytic enzyme or cellulase: The term “cellulolytic enzyme” or “cellulase” means one or more (e.g., at least one, several) enzymes that hydrolyze a cellulosic material. Cellulases have been traditionally divided into three major classes: endoglucanases (EC 3.2.1.4) (“EG”), exoglucanases or cellobiohydrolases (EC 3.2.1.91) (“CBH”) and beta-glucosidases ([beta]-D-glucoside glucohydrolase; EC 3.2.1.21) (“BG”). (Knowles et al., 1987; Shulein, 1988). Endoglucanases act mainly on the amorphous parts of the cellulose fiber, whereas cellobiohydrolases are also able to degrade crystalline cellulose (Nevalainen and Penttila, 1995). Thus, the presence of a cellobiohydrolaase in a cellulase system is typically required for most efficient solubilization of crystalline cellulose (Suurnakki, et al. 2000). Beta-glucosidase acts to liberate D-glucose units from cellobiose, cello-oligosaccharides, and other glucosides (Freer, 1993). Total cellulolytic activity may be measured using insoluble substrates, including Whatman N^{o} 1 filter paper, microcrystalline cellulose, algal cellulose, cotton, pretreated lignocellulose, etc. The most common total cellulolytic activity assay is the filter paper assay using Whatman N^{o} 1 filter paper as the substrate established by the International Union of Pure and Applied Chemistry (IUPAC) (Ghose, 1987, Measurement of cellulase activities, Pure Appl. Chem. 59: 257-68).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Solvent(s): The term “solvent,” or “cellulosic solvent(s)”, as used herein, means any solvent or combination of solvents that is capable of disrupting the structure of the cellulose-hemicellulose-lignin matrix of a cellulosic material. The particular mechanism by which the cellulosic solvent effects disruption (e.g., dissolving, swelling, plasticizing, or reorganizing cellulose, hemicellulose, lignin, or any portion of the biomass) is not critical to the methods and processes described herein so long as the solvent disrupts the structure of the cellulose-hemicellulose-lignin matrix. Preferably, the solvent is a cellulose solvent that disrupts the structure of the matrix to cause the cellulosic material to be more readily hydrolyzable. (e.g., by enzymatic hydrolysis, etc.).

Effective amount(s): The terms “effective amount” and “effective concentration” as used herein, mean the amount or concentration of at least one solvent, such as a cellulosic solvent (e.g., at least one polyhydric alcohol) that is sufficient to cause a desired improvement in a treatment process (e.g., a cellulosic pretreatment process.) The actual effective amount in absolute value depends on factors including, but not limited to, the cellulosic solvent or combination of cellulosic solvents used, the cellulosic material to be treated, the size (e.g., volume, etc.) of the vessel used in the treatment process, and/or synergistic or antagonistic interactions between treatment agents, which may increase or reduce the efficiency of the pretreatment process (e.g., increase or reduce the solvolysis of a cellulosic material subjected to a pretreatment process). The “effective amount” or “effective concentration” of the at least one cellulosic solvent may be determined, e.g., by a routine dose response experiment.

Fermentable sugar(s): The term “fermentable sugar(s)”, as used herein, refers to oligosaccharides and monosaccharides that can be used as a carbon source by a microorganism in a fermentation process.

Fractionation: The terms “fractionation” or “fractionated”, as used herein, means the removal or separation of at least some portion of biomass, such as cellulose from a cellulosic material or a lignocellulosic containing material.

Hemicellulose: The term “hemicellulose”, as used herein, means an oligosaccharide or polysaccharide of biomass material other than cellulose. Hemicellulose is chemically heterogeneous and includes a variety of polymerized sugars, primarily D-pentose sugars, such as xylans, xyloglucans, arabinoxylans, and mannans, in complex heterogeneous branched and linear polysaccharides or oligosaccharides that are bound via hydrogen bonds to the cellulose microfibrils in the plant cell wall, and wherein xylose sugars are usually in the largest amount. Hemicelluloses may be covalently attached to lignin, and usually hydrogen bonded to cellulose, as well as to other hemicelluloses, which help stabilize the cell wall matrix forming a highly complex structure. Hemicellulosic material includes any form of hemicellulose, such as polysaccharides degraded or hydrolyzed to oligosaccharides. It is understood herein that the hemicellulose may be in the form of a component of lignocellulose, a plant cell wall material containing lignin, cellulose, and hemicellulose in a mixed matrix.

Hemicellulolytic enzyme or hemicellulase: The term “hemicellulolytic enzyme” or “hemicellulase” means a class of enzymes capable of breaking hemicellulose into its component sugars or shorter polymers, and includes endo-acting hydrolases, exo-acting hydrolases, and various esterases. Non-limiting examples of hemicellulases include, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. Classification of these and other carbohydrate active enzymes is available in the Carbohydrate-Active Enzymes (CAZy) database. Hemicellulolytic enzyme activities may be measured according to Ghose and Bisaria, 1987, Pure & Appl. Chem. 59: 1739-1752, at a suitable temperature, e.g., 50° C., 55° C., or 60° C., and pH, e.g., 5.0 or 5.5.

Hydrolysis: The terms “hydrolysis”, “hydrolyze”, and/or “digestion”, as used herein, may be used interchangeably and means to cleave a polymer under the action of acid, enzyme, heat, shear, or combination thereof. The mode and rate of hydrolysis, and therefore the composition of the resulting product, is related to the type of enzyme used, the concentration of substrate present, and exposure time, etc.

Lignin: The term “lignin” means a complex chemical compound most commonly derived from wood and generally being an integral part of the secondary cell walls of plants.

Ligninolytic enzyme: The term “ligninolytic enzyme” means an enzyme that hydrolyzes the structure of lignin polymers. Enzymes that can break down lignin include lignin peroxidases, manganese peroxidases, laccases and feruloyl esterases, and other enzymes described in the art known to depolymerize or otherwise break lignin polymers. Also included are enzymes capable of hydrolyzing bonds formed between hemicellulosic sugars (notably arabinose) and lignin.

Lignocellulose-containing material(s): The terms “lignocellulose-containing material(s)”, “lignocellulosic containing material(s)”, and/or “lignocellulosic material(s)” as used herein means any material that primarily consists of cellulose, hemicellulose, and lignin. The terms “lignocellulose-containing material(s)”, “lignocellulosic containing material(s)”, and/or “lignocellulosic material(s)” may be used interchangeably.

Polyhydric alcohol(s): The term “polyhydric alcohol(s)” as used herein has its conventional meaning to one skilled in the art and means the reduction product of sugars wherein the carbonyl group has been reduced to an alcohol. The term “polyhydric alcohol(s)” may be used interchangeably with the terms “polyalcohol(s)”, “glycitol(s)” and/or “sugar alcohol(s)”.

Pretreatment: The terms “pretreatment” or “pretreatment process(es)” as used herein may be used interchangeably and means any treatment intended to separate and/or release cellulose, hemicellulose, and/or lignin from a cellulosic material. Any pretreatment process can be used to disrupt plant cell wall components of the cellulosic material (Chandra et al., 2007, Substrate pretreatment: The key to effective enzymatic hydrolysis of lignocellulosics, Adv. Biochem. Eng. Biotechnol. 108: 67-93; Galbe and Zacchi, 2007, Pretreatment of lignocellulosic materials for efficient bioethanol production, Adv. Biochem. Engin./Biotechnol. 108: 41-65; Hendriks and Zeeman, 2009, Pretreatments to enhance the digestibility of lignocellulosic biomass, Bioresource Technology 100: 10-18; Mosier et al., 2005, Features of promising technologies for pretreatment of lignocellulosic biomass, Bioresource Technology 96: 673-686; Taherzadeh and Karimi, 2008, Pretreatment of lignocellulosic wastes to improve ethanol and biogas production: a review, Int. J. Mol. Sci. 9: 1621-1651; Yang and Wyman, 2008, Pretreatment: the key to unlocking low-cost cellulosic ethanol, Biofuels Bioproducts and Biorefining-Biofpr. 2: 26-40).

Reactor: The terms “reactor” or “pretreatment reactor” as used herein may be used interchangeably and mean any vessel suitable for practicing a method of the present invention. The dimensions of the reactor should be sufficient to accommodate the materials conveyed into and out of the reactor (e.g., lignocellulosic containing materials, solvents, etc.), as well as additional headspace around the material. Furthermore, the reactor should be constructed of materials capable of withstanding the subject conditions (e.g., conditions required for the pretreatment of a lignocellulosic containing material) and the reactor should be such that conditions (e.g., pH, temperature, pressure, etc.) do not affect the integrity or performance of the vessel. In the context of this disclosure, the term reactor may be used interchangeably with mixer, or melt compounding equipment, or extruder.

Saccharification: The term “saccharification” as used herein refers to the production of fermentable sugars from polysaccharaides. The term “partial saccharification” as used herein refers to the limited saccharification of a cellulosic material wherein the fermentable sugars released are less than the total fermentable sugars that would be released if saccharification is run to completion.

Slurry: The term “slurry” as used herein means the cellulosic material that undergoes enzymatic hydrolysis. A slurry (e.g., a biomass slurry) is produced by mixing cellulosic material, with a solvent (e.g., water, at least one cellulosic solvent such as at least one polyhydric alcohol, etc.) and/or other pre-treatment materials.

Unhydrolyzed Solid(s) or Unconverted Solids: The terms “unhydrolyzed solids” or “unconverted solids” may be used interchangeably and means cellulosic material that is not digested by a cellulose hydrolyzing enzyme (e.g. a cellulase), as well as non-cellulosic or other, materials that are inert to a cellulose hydrolyzing enzyme. Non-limiting examples of unconverted solids may include lignin, silica or other solid material. As the cellulose is hydrolyzed, the concentration of unconverted solids within the cellulose-containing solid particles increases.

Described herein is a highly efficient biomass pretreatment method that avoids toxic chemicals and corrosive acids/bases yielding a simple, scalable process to fractionate biomass using existing low-cost equipment and provides a recoverable superior lignin co-product.

Cellulosic Material:

According to the methods described herein, the biomass or cellulosic material may be any material comprising cellulosic fibers. Examples of such materials include, but are not limited to, wood, straw, hay, grass, silage, such as cereal silage, corn silage, grass silage; bagasse, etc. A suitable material comprising cellulosic fibers is crop stover, (e.g., corn stover). Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York).

In an embodiment, the cellulosic material is any biomass material. In another aspect, the cellulosic material is lignocellulose (e.g., a lignocellulosic biomass), a plant cell wall material containing lignin, cellulose, and hemicellulose in a mixed matrix. Lignocellulosic containing material is generally found, for example, in the stems, leaves, hulls, husks, and cobs of plants or leaves, branches, and wood of trees. Lignocellulosic material can also be, but is not limited to, herbaceous material, agricultural residues/sidestreams (e.g., corn stover, corn fiber, soybean stover, soybean fiber, tobacco stover, tobacco midrib, tobacco fiber, rice straw, pine wood, wood chips, poplar, wheat straw, switchgrass, bagasse, etc.), materials traditionally used for silaging (e.g., green chopped whole corn, hay, alfalfa, etc.), forestry residues, municipal solid wastes, waste paper, and pulp and paper mill residues.

In one aspect, the cellulosic material is an agricultural residue. In another aspect, the cellulosic material is herbaceous material (including energy crops). In another aspect, the cellulosic material is municipal solid waste. In another aspect, the cellulosic material is pulp and paper mill residue. In another aspect, the cellulosic material is waste paper. In another aspect, the cellulosic material is wood (including forestry residue). In a more particular aspect the wood is selected from the group consisting of Liquidambar styraciflua (i.e., American Sweetgum), Senegalia (Acacia) senegal, Vachellia (Acacia) seyal, and combinations thereof. In an even more particular aspect, the wood is Liquidambar styraciflua (i.e., American Sweetgum).

In another aspect, the cellulosic material is arundo. In another aspect, the cellulosic material is bagasse. In another aspect, the cellulosic material is bamboo. In another aspect, the cellulosic material is corn cob. In another aspect, the cellulosic material is corn fiber. In another aspect, the cellulosic material is corn stover. In another aspect, the cellulosic material is miscanthus. In another aspect, the cellulosic material is orange peel. In another aspect, the cellulosic material is rice straw. In another aspect, the cellulosic material is switchgrass. In another aspect, the cellulosic material is wheat straw. In another aspect the cellulosic material is tobacco stover. In another aspect the cellulosic material is tobacco midrib (e.g. tobacco stem). In another aspect the cellulosic material is tobacco fiber.

In another aspect, the cellulosic material is spent grain. As used herein, the term “spent grain” means a range of grains and cereals that are byproducts of the brewing and distilling processes. Non-limiting examples include wheat, barley, rye, corn, millet, and sorghum. In another aspect, the cellulosic material is grain that that has been used in the brewing or distillation of alcohol. In another aspect, the cellulosic material is wheat grain that that has been used in the brewing or distillation of alcohol. In another aspect, the cellulosic material is barley grain that that has been used in the brewing or distillation of alcohol. In another aspect, the cellulosic material is rye grain that that has been used in the brewing or distillation of alcohol. In another aspect, the cellulosic material is corn grain that that has been used in the brewing or distillation of alcohol. In another aspect, the cellulosic material is millet grain that that has been used in the brewing or distillation of alcohol. In another aspect, the cellulosic material is sorghum grain that that has been used in the brewing or distillation of alcohol.

In another aspect, the cellulosic material is aspen. In another aspect, the cellulosic material is eucalyptus. In another aspect, the cellulosic material is fir. In another aspect, the cellulosic material is pine. In another aspect, the cellulosic material is poplar. In another aspect, the cellulosic material is spruce. In another aspect, the cellulosic material is willow.

In another aspect, the cellulosic material is algal cellulose. In another aspect, the cellulosic material is cotton linter. In another aspect, the cellulosic material is filter paper. In another aspect, the cellulosic material is microcrystalline cellulose.

In another aspect, the cellulosic material is an aquatic biomass. As used herein the term “aquatic biomass” means biomass produced in an aquatic environment by a photosynthesis process.

Pretreatment of Biomass or Cellulosic Materials:

According to aspects and embodiments of the methods described herein, the pretreating step can be any pretreating step known in the art for the pretreatment of cellulosic materials. Non-limiting examples of conventional cellulosic material pretreatments include, but are not limited to, heat pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

In a particular aspect, the pretreatment step is a heat pretreatment (i.e., a thermolytic treatment that promotes thermolysis). Thermolysis, as used herein, means bringing about any chemical change in a substance (e.g., cellulosic materials, lignocellulosic materials, etc.) through the application of heat. In particular aspects, the heat pretreatments may further comprise subjecting the cellulosic material to one or more solvents for a period of time (e.g., 1 to 120 minutes) at various high heat temperatures between 100° C. and 300° C. At these temperatures, the heat pretreatment is used to heat and shear polymeric materials (i.e., separate cellulose, hemicellulose, lignin, and other oligosaccharides present in the cellulosic material).

In certain aspects pretreatment is performed at temperatures in the range from equal to or more than 100° C. to equal to or less than 300° C. In a particular aspect, the pretreatment is performed at temperatures in the range from equal to or more than 200° C. to equal to or less than 300° C. e.g., 200° C., 210° C., 220° C., 230° C., 240° C., 250° C., 260° C., 270° C., 280° C., 290° C., 300° C., where the optimal temperature range depends on various factors, including, but not limited to, the amount of cellulosic material to be pretreated, the amount of cellulosic solvent to be used, residence time, etc. In a more particular aspect, the pretreatment temperature is 200° C. In still a more particular aspect, the pretreatment temperature is 240° C.

Residence times of the various steps are also not regarded as critical, provided that the intended function is accomplished. In a particular aspect, the residence time for a particular heat pretreatment may range from equal to or more than 1 minute to equal to or less than 120 minutes. In a particular aspect, the residence time for the pretreatment step ranges from equal to or more than 1 minute to equal to or less than 15 minutes. In a more particular aspect, the residence time for the pretreatment step ranges from equal to or more than 4 minutes to equal to or less than 12 minutes. In a particular aspect, the residence time for the pretreatment step is about 8 minutes.

In another aspect, the heat pretreatment is performed in the presence of at least one cellulosic solvent. In a particular aspect, the heat pretreatment is performed in the presence of more than one cellulosic solvent (e.g., at least two cellulosic solvents, at least three cellulosic solvents, at least four cellulosic solvents, at least five cellulosic solvents, at least six cellulosic solvents, at least seven cellulosic solvents, at least eight cellulosic solvents, at least nine cellulosic solvents, at least ten cellulosic solvents, etc.). In a particular aspect, two or more cellulosic solvents can be used simultaneously or piece-meal at appropriate times as determined by the process or method being performed.

In a particular aspect, the at least one cellulosic solvent is at least one polyhydric alcohol. The form of the polyhydric alcohol is not critical, and may take any form so long as the polyhydric alcohol is suitable for practicing the methods and processed described herein. For example, the polyhydric alcohol may be employed as a solid (e.g., crystalline) polyhydric alcohol; a liquid (e.g., a syrup); an aqueous mixture (e.g., a mixture of water and a polyhydric alcohol); a non-aqueous mixture of an organic solvent and polyhydric alcohol (e.g., acetone and a polyhydric alcohol); or any combination thereof.

In a more particular aspect, the at least one polyhydric alcohol is a polyhydric alcohol having from 1 to 60 carbon atoms and having from 1 to 60 hydroxyl groups. In another aspect, the at least one polyhydric alcohol is a polyhydric alcohol having from 1 to 6 carbon atoms and having from 1 to 4 hydroxyl groups. In still yet a more particular aspect, the at least one polyhydric alcohol is a polyhydric alcohol having from 2 to 4 carbon atoms and having from 2 to 3 hydroxyl groups.

Non-limiting examples of at least one polyhydric alcohol that may be used according to the processes and methods described herein include, various propanediols, various dipropanediols, various tripropanediols, various butanediols, various dibutanediols, various pentanediols, various pentanetriols, various hexanediols, various hexanetriols, various cyclohexanediols, various cyclohexanetriols, pentaerythritols, and combinations thereof.

Specific examples of the at least one polyhydric alcohol that may be used according to the processes and methods described herein include, but are not limited to, 1,6-anhydro-glucose, 2,5-anhydro-D-mannitol, 1,2,6-hexanetriol, arabitol, adonitol, butanetriol, dulcitol, diethylene glycol, diglycerol, erythritol, ethanol, ethylene glycol, fucitol, galactol, glycerol, iditol, inositol, isomalt, lacitol, maltitol, maltotetraitol, maltotriitol, mannitol, mesoerythritol, methanol, polyethylene glycol, polyglycitol, polyglycerol, ribitol, scyllitol, sorbitol, triethylene glycol, triglycerol, trimethylolpropane, threitol, volemitol, xylitol, and combinations thereof.

In a particular aspect, the at least one polyhydric alcohol is chosen from at least one of 1,6-anhydro-glucose, 2,5-anhydro-D-mannitol, 1,2,6-hexanetriol, arabitol, adonitol, butanetriol, dulcitol, diethylene glycol, diglycerol, erythritol, ethanol, ethylene glycol, fucitol, galactol, glycerol, iditol, inositol, isomalt, lacitol, maltitol, maltotetraitol, maltotriitol, mannitol, mesoerythritol, methanol, polyethylene glycol, polyglycitol, polyglycerol, ribitol, scyllitol, sorbitol, triethylene glycol, triglycerol, trimethylolpropane, threitol, volemitol, xylitol, and combinations thereof.

In one aspect, the at least one polyhydric alcohol is glycerol.

In yet another aspect, the heat pretreatments according to the processes and methods described herein may be a heat pretreatment performed through a hot-melt extrusion process (e.g., a melt compounding process). Hot melt-extrusion, or melt compounding, is a process understood to those skilled in the art and is intended to describe the process of efficiently heating and shearing polymeric material in industrial equipment (e.g., melt compounders, extruders, etc.). Melt compounding equipment (e.g., melt compounders, micro-compounders extruders, etc.) is well known in the art and widely used in the polymer industry to process 100,000 billion pounds of material a year in a continuous process.

Use of melt compounding equipment for heat pretreatment is beneficial for certain embodiments of the described methods disclosed herein. In particular, melt compounding is scalable, (i.e., melt compounding can be performed at a rate of up to about 10¹ kg/hr and up to 10⁴ kg/hr depending on the processing conditions), is common and easily available, and modular (i.e., extruders have interchangeable screws and screw elements that allow for spatial control of pressure during the process and spatial control of temperature and solvent composition through venting).

In particular aspects of the methods and processes described herein, cellulosic material is heat treated processed using melt compounding processes and machinery. It is envisioned that the melt compounding process can be performed at relatively high solids loading (e.g., about 25% w/w to about 50% w/w compared to conventional pretreatments between about 5% w/w to about 20% w/w).

In a more particular aspect of the methods and processes described herein, the heat pretreatment processes is a hot-melt extrusion process, or a melt compounding process, in the presence of at least one cellulosic solvent. In still a more particular aspect, the at least one cellulosic solvent is at least one polyhydric alcohol. In a more particular aspect the at least one polyhydric alcohol is glycerol.

Enzymatic Hydrolysis:

In aspects of the processes and methods described herein, enzymatic digestion or enzymatic degradation of pretreated cellulosic material is the same as hydrolyzing a pretreated cellulosic material.

Suitable method conditions for the enzymatic hydrolysis of a cellulosic material are well-known to the skilled artisan or can easily be determined by a person skilled in the art. In a particular aspect, the enzymatic hydrolysis is of a cellulosic material, wherein the cellulosic material has been pretreated according to one or more of the pretreatment methods described in this disclosure.

The enzymatic hydrolysis reaction may continue until the desired level of hydrolysis of the cellulosic material has been achieved. The progress of enzyme reaction may be measured by various methods. If specific parameters have been established for achieving a particular composition, then the reaction may be allowed to proceed to a predetermined relative end point in time. The end point also may be monitored and defined by measuring the concentration of reducing sugars. Other techniques such as monitoring the change in viscosity, spectral changes, or the change in molecular weight may be used to define the reaction end point.

The hydrolysis reaction may be carried out for periods ranging from a few minutes to many hours or more depending on the temperature (or temperatures of the reaction), pressure (or pressures inside the reactor during the reaction), enzyme (or enzymes, or enzyme suites) used in the reaction, substrate concentrations of the reaction, and other variables. The enzyme action may then be terminated by means well-known to the skilled artisans (e.g., heat, chemical additions, or other methods known in the art for deactivating an enzyme or separating an enzyme from its substrate).

In an aspect, enzymatic hydrolysis may be carried out at 10-50% (w/w) TS (Total Solids), such as at 15-40% TS, such as at 15-30% TS, such as at around 20% TS. In a particular aspect, enzymatic hydrolysis is carried out at 20-50% TS. Hydrolysis of the cellulosic material may be carried out for 12-240 hours, such as for 24-192 hours, such as for 48-144 hours, such as for around 96 hours, such as for around 72 hours, such as for around 48 hours, such as for around 24 hours, such as for around 18 hours, such as for around 12 hours, etc. The temperature during hydrolysis may be between 30-70° C., such as 40-60° C., such as 45-55° C., such as around 50° C. The pH during hydrolysis may be between 4-7, such as pH 4.5-6, such as around pH 5.

Suitable enzymes for use in the enzymatic hydrolysis of a cellulosic material include at least one enzyme capable of hydrolyzing a cellulosic material. Non-limiting examples of enzymes capable of hydrolyzing (i.e., degrading, digesting, etc.) a cellulosic material include, cellulolytic enzymes, hemicellulolytic enzymes, ligninolytic enzymes, and combinations thereof.

Specific enzymes that may be useful for some aspects of the processes and methods disclosed herein include one or more enzymes selected from the group consisting of amylases, carbohydrases, catalases, cellulases, beta-glucanases, glucuronidases, hemicellulases, laccases, ligninolytic enzymes, lipases, pectinases, peroxidases, phytases, proteases, swollenins, and/or any combination thereof, including more than two, such as, at least three of the above enzymes, at least four of the above enzymes, at least five of the above enzymes, at least six of the above enzymes, at least seven of the above enzymes, at least eight of the above enzymes, at least nine of the above enzymes, at least ten of the above enzymes, at least eleven of the above enzymes, at least twelve of the above enzymes, at least thirteen of the above enzymes, at least fourteen of the above enzymes, up to and including all of the above enzymes.

In another aspect, the at least one enzyme for use in the enzymatic hydrolysis of a cellulosic material comprises at least one (e.g., several) cellulolytic enzyme. In another aspect, the at least one enzyme for use in the enzymatic hydrolysis of a cellulosic material comprises at least one (e.g., several) hemicellulolytic enzyme. In another aspect, the at least one enzyme for use in the enzymatic hydrolysis of a cellulosic material comprises at least one (e.g., several) ligninolytic enzyme. In another aspect, the at least one enzyme for use in the enzymatic hydrolysis of a cellulosic material comprises at least one (e.g., several) enzyme selected from the group of cellulolytic enzymes, hemicellulolytic enzymes, and ligninolytic enzymes.

In a particular aspect, the at least one enzyme for use in the enzymatic hydrolysis of a cellulosic material comprises at least one cellulase. In a more particular aspect, the at least one cellulase is at least one cellulase selected from the group consisting of an endoglucanase, a cellobiohydrolase, and a beta-glucosidase. Non-limiting examples of commercial cellulolytic enzyme preparations suitable for use in the processes and methods described herein include, for example, CELLIC® CTec (Novozymes A/S), CELLIC® CTec2 (Novozymes A/S), CELLIC® CTec3 (Novozymes A/S), CELLUCLAST™ (Novozymes A/S), NOVOZYM™ 188 (Novozymes A/S), CELLUZYME™ (Novozymes A/S), CEREFLO™ (Novozymes A/S), and ULTRAFLO™ (Novozymes A/S), ACCELERASE™ (Genencor Int.), LAMINEX™ (Genencor Int.), SPEZYME™ CP (Genencor Int.), FILTRASE® NL (DSM); METHAPLUS® S/L 100 (DSM), ROHAMENT™ 7069 W (Rohm GmbH), FIBREZYME® LDI (Dyadic International, Inc.), FIBREZYME® LBR (Dyadic International, Inc.), or VISCOSTAR® 150L (Dyadic International, Inc.).

In another aspect, the at least one enzyme for use in the enzymatic hydrolysis of a cellulosic material comprises at least one hemicellulase. In a more particular embodiment, the at least one hemicellulase is at least one hemicellulase selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. Non-limiting examples of commercial hemicellulolytic enzyme preparations suitable for use in the processes and methods disclosed herein include, for example, SHEARZYME™ (Novozymes A/S), CELLIC® HTec (Novozymes A/S), CELLIC® HTec2 (Novozymes A/S), CELLIC® HTec3 (Novozymes A/S), VISCOZYME® (Novozymes A/S), ULTRAFLO® (Novozymes A/S), PULPZYME® HC (Novozymes A/S), MULTIFECT® Xylanase (Genencor), ACCELLERASE® XY (Genencor), ACCELLERASE® XC (Genencor), ECOPULP® TX-200A (AB Enzymes), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK).

### Methods of the Disclosed Embodiments

In an embodiment methods described herein disclose a process for pretreating a cellulosic material for enzymatic hydrolysis, comprising:

a. mixing at least one cellulosic material and at least one cellulosic solvent in a reactor to form a biomass slurry; and

b. heating the biomass slurry at a temperature in the range from equal to or more than 100° C. to equal to or less than 300° C. to obtain a pretreated cellulosic material for enzymatic hydrolysis.

Particular methods include a method of processing lignocellulosic biomass, comprising: providing lignocellulosic biomass and at least one solvent; providing and heating a mixer to a temperature of between 100° C. to 300° C.; adding the biomass and the solvent to the mixer; mixing the biomass and solvent into a biomass slurry; and melt compounding the biomass slurry under shearing and heating for an amount of time to cause disruption of inter- or intra-polymer linkages of the biomass.

For example, methods can include processing lignocellulosic biomass by mixing lignocellulosic biomass and glycerol to form a biomass slurry; and heating and shearing the biomass slurry at a temperature ranging from 100° C. to 300° C. for an amount of time to disrupt inter- or intra-polymer linkages of the biomass.

In certain aspects of the method, biomass is processed to obtain a cellulosic material, wherein the method fractionates at least 10% of the cellulose present (i.e., at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or more, such as all, of the cellulose present) from the at least one cellulosic material. In a particular aspect, a pretreated cellulosic material is obtained, wherein the pretreatment fractionates at least 50% of the cellulose present from the at least one cellulosic material. In a more particular aspect, a pretreated cellulosic material is obtained, wherein the pretreatment fractionates at least 60% of the cellulose present from the at least one cellulosic material. In still a more particular aspect, a pretreated cellulosic material is obtained, wherein the pretreatment fractionates at least 70% of the cellulose present from the at least one cellulosic material. In yet a more particular aspect, a pretreated cellulosic material is obtained, wherein the pretreatment fractionates at least 80% of the cellulose present from the at least one cellulosic material. In still yet a more particular aspect, a pretreated cellulosic material is obtained, wherein the pretreatment fractionates at least 90% of the cellulose present from the at least one cellulosic material. In a more particular aspect, a pretreated cellulosic material is obtained, wherein the pretreatment fractionates at least 95% of the cellulose present from the at least one cellulosic material. In still yet a more particular aspect, a pretreated cellulosic material is obtained, wherein the pretreatment fractionates at least 99% of the cellulose present from the at least one cellulosic material.

In aspects of the method disclosed, the at least one cellulosic material and the at least one cellulosic solvent may be mixed in a reactor to form a slurry before, after, or simultaneously with the heating step.

In one aspect, the at least one cellulosic material and the at least one cellulosic solvent are mixed in a reactor to form a slurry before the heating step. In a particular aspect the at least one cellulosic material and the at least one cellulosic solvent can be mixed in a separate reactor to form a slurry and then transferred to a reactor for the heating step. In another aspect the at least one cellulosic material and the at least one cellulosic solvent can be mixed simultaneously to form a slurry in the reactor that will be used for the heating step, but mixed prior to heating.

In another aspect, the at least one cellulosic material and the at least one cellulosic solvent are mixed in a reactor to form a slurry after the heating step. In a particular aspect, the at least one cellulosic material and the at least one cellulosic solvent can be mixed in a separate reactor to form a slurry and then transferred to a preheated reactor for the heating step.

In yet another aspect, the at least one cellulosic material and the at least one cellulosic solvent are mixed in a reactor to form a slurry simultaneously with the heating step. In a particular aspect, the at least one cellulosic material and the at least one cellulosic solvent can be mixed in a separate reactor (e.g., a preheated reactor) to form a preheated slurry and then transferred to a preheated reactor for the heating step. In another particular aspect, the at least one cellulosic material and the at least one cellulosic solvent can be mixed to form a slurry in a reactor as the reactor is simultaneously heated for the heating step.

In particular aspects of the method disclosed, the biomass slurry is heated to a temperature in the range from equal to or more than 200° C. to equal to or less than 250° C. In a particular aspect, the biomass slurry is heated to a temperature of about 200° C. (i.e., 200° C.). In another particular aspect, the biomass slurry is heated to a temperature of about 240° C. (i.e., 240° C.).

In aspects of the method disclosed, the residence time of the biomass slurry in the reactor may range from equal to or more than 10 seconds to equal to or less than 24 hours. In a particular aspect, the residence time of the biomass slurry in the reactor ranges from equal to or more than 2 minutes to equal to or less than 15 minutes. In a more particular aspect, the residence time of the biomass slurry in the reactor ranges from equal to or more than 4 minutes to equal to or less than 12 minutes. In still an even more particular aspect, the residence time of the biomass slurry in the reactor is 8 minutes.

In aspects of methods disclosed, the cellulosic material is a lignocellulosic material. In embodiments the cellulosic material is a lignocellulosic material selected from the group consisting of wood (including forestry residue), agricultural residue, spent grains, and combinations thereof. In a more particular cellulosic material is selected from the group consisting of Liquidambar styraciflua (i.e., American Sweetgum), Senegalia (Acacia) senegal, Vachellia (Acacia) seyal, corn cob, corn fiber, corn stover, tobacco stover, tobacco midrib, tobacco fiber, spent grain, orange peel, and combinations thereof.

In aspects, the ratio of cellulosic material to cellulosic solvent is present in a weight ratio of from 1:100 to 100:1, such as from 1:50 to 50:1, or from 1:25 to 25:1, or from 1:10 to 10:1, or from 1:5 to 5:1 or from 1:2 to 2:1, or about 1:1.

In another aspect, the at least one cellulosic solvent is at least one polyhydric alcohol. In another aspect, the at least one polyhydric alcohol is at least one polyhydric alcohol having from 1 to 60 carbon atoms and having from 1 to 60 hydroxyl groups. In still another aspect, the at least one polyhydric alcohol is at least one polyhydric alcohol having from 1 to 6 carbon atoms and having from 1 to 4 hydroxyl groups. In still yet another aspect, the at least one polyhydric alcohol is a polyhydric alcohol having from 2 to 4 carbon atoms and having from 2 to 3 hydroxyl groups. In a particular aspect, the at least one polyhydric alcohol is selected from the group consisting of 1,6-anhydro-glucose, 2,5-anhydro-D-mannitol, 1,2,6-hexanetriol, arabitol, adonitol, butanetriol, dulcitol, diethylene glycol, diglycerol, erythritol, ethanol, ethylene glycol, fucitol, galactol, glycerol, iditol, inositol, isomalt, lacitol, maltitol, maltotetraitol, maltotriitol, mannitol, mesoerythritol, methanol, polyethylene glycol, polyglycitol, polyglycerol, ribitol, scyllitol, sorbitol, triethylene glycol, triglycerol, trimethylolpropane, threitol, volemitol, xylitol, and combinations thereof. In a more particular aspect, the at least one polyhydric alcohol is glycerol.

In various aspects, additional steps may be performed and within the scope of the method disclosed. In a particular aspect, the method includes the further step of recovering the pretreated cellulosic material.

In an aspect, recovering the pretreated cellulosic material may further comprise extraction of lignin. Extraction of lignin can be performed according to conventional means known to those skilled in the art (e.g., filtering, gravity setting, decanting, centrifuging, hydrocyclone separation, or combinations thereof). In a particular aspect, after heat pretreatment, the lignin can be precipitated and isolated according to methods known in the art. In a particular aspect, the extraction may be repeated as necessary (e.g., the extraction step may be performed at least two times, at least three times, at least four times, at least five times, at least six time, at least seven times, at least eight times, at least nine times, at least ten times, and so on). In a particular aspect, at least 10% of the lignin is extracted from the pretreated cellulosic material (i.e., at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or more, such as all, of the lignin is extracted from the pretreated cellulosic material).

In another aspect, recovering the pretreated cellulosic material may further comprise extraction of xylan. Again, extraction of xylan can be performed according to conventional means known to those skilled in the art (e.g., filtering, gravity setting, chromatography (e.g. column chromatography), decanting, centrifuging, hydrocyclone separation, or combinations thereof). In a particular aspect, after heat pretreatment, the xylan can be precipitated and isolated according to methods known in the art. In a particular aspect, the extraction may be repeated as necessary (e.g., the extraction step may be performed at least two times, at least three times, at least four times, at least five times, at least six time, at least seven times, at least eight times, at least nine times, at least ten times, and so on). In a particular aspect, at least 10% of the xylan is extracted from the pretreated cellulosic material (i.e., at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or more, such as all, of the xylan is extracted from the pretreated cellulosic material).

In another aspect, recovering the pretreated cellulosic material may further include washing and/or isolating the pretreated cellulosic material. Washing may be performed before or after lignin extraction and then may be repeated as necessary (e.g., the washing step may be performed at least two times, at least three times, at least four times, at least five times, at least six time, at least seven times, at least eight times, at least nine times, at least ten times, and so on). The washing step may include washing the pretreated cellulosic material with one or more solvents known to the skilled artisan (e.g., at least two solvents, at least three solvents, at least four solvents, at least five solvents, at least six solvents, at least seven solvents, at least eight solvents, at least nine solvents, at least ten solvents, and so on). Non-limiting examples of solvents for washing include water, methanol, ethanol, sodium hydroxide, etc.

Solvents used for washing may be separated from the washed pretreated cellulosic material by any suitable means. Non-limiting examples include filtering, gravity setting, chromatography (e.g. column chromatography), decanting, centrifuging or hydrocyclone separation.

In still another aspect, the method further comprises the step of drying. In a preferred method, the product from the process is filtered, washed, and then dried appropriately. Conventional drying methods are known in the art (e.g., air-drying, vacuum drying, rotary evaporation, etc.). The drying may occur at any point in the method described and may be repeated as necessary (e.g., the drying step may be performed at least two times, at least three times, at least four times, at least five times, at least six time, at least seven times, at least eight times, at least nine times, at least ten times, and so on). In aspects of the method described, the drying time will vary and will be determined based on whether the pretreated cellulosic material is adequately or substantially dry. In a particular embodiment, the pretreated cellulosic material is dried up to about 72 hours, such from 24-48 hours, or up to 8 hours, or up to 4 hours, etc.

In still another aspect, the method further comprises the step of bleaching. In a preferred method, the product from the process is bleached. Bleaching can be performed during any stage in the process and may occur more than once (e.g., at least two, at least three times, at least four times, at least five times, at least six times, at least seven times, at least eight time, at least nine times, at least ten times, and so on). Conventional bleaching methods are known in the art (e.g., with hydrogen peroxide, sodium hydroxide, etc. In aspects of the method described, the amount of bleaching and the duration of bleaching will vary and will be determined based on product following the pretreatment process.

In yet another embodiment, methods described herein disclose a process for hydrolyzing a cellulosic material comprising:

a. mixing at least one cellulosic material and at least one cellulosic solvent in a reactor to form a slurry;

b. heating and shearing the slurry at a temperature in the range from equal to or more than 100° C. to equal to or less than 300° C. to obtain a pretreated cellulosic material;

c. and hydrolyzing the pretreated cellulosic material with at least one enzyme capable of converting the pretreated cellulosic material into at least one fermentable sugar.

Aspects of steps a. and b. can be carried out as described above and the method may or may not further comprise one or more additional steps (e.g., recovery, washing, drying, etc.). Hydrolysis may be performed according to the “Hydrolysis” section detailed above. Conditions for hydrolysis may vary depending on a number of factors including enzyme, or combinations of enzymes and/or enzyme suites used, whether the saccharification is run to completion, and the amount of fractionated cellulose present in the pretreated cellulosic material.

In a particular aspect, hydrolyzing the pretreated cellulosic material converts at least 10% of the pretreated cellulosic material (i.e., at least 10%, at least 20%, at least 30%, at least 40%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or more, such as all, of the pretreated cellulosic material) to at least one fermentable sugar. In a particular aspect, hydrolyzing the pretreated cellulosic material converts at least 50% of the pretreated cellulosic material to at least one fermentable sugar. In a more particular aspect, hydrolyzing the pretreated cellulosic material converts at least 60% of the pretreated cellulosic material to at least one fermentable sugar. In still a more particular aspect, hydrolyzing the pretreated cellulosic material converts at least 70% of the pretreated cellulosic material to at least one fermentable sugar. In yet a more particular aspect, hydrolyzing the pretreated cellulosic material converts at least 80% of the pretreated cellulosic material to at least one fermentable sugar. In still yet a more particular aspect, hydrolyzing the pretreated cellulosic material converts at least 90% of the pretreated cellulosic material to at least one fermentable sugar. In a more particular aspect, hydrolyzing the pretreated cellulosic material converts at least 95% of the pretreated cellulosic material to at least one fermentable sugar. In still yet a more particular aspect, hydrolyzing the pretreated cellulosic material converts at least 99% of the pretreated cellulosic material to at least one fermentable sugar.

In a more particular embodiment, hydrolyzing the pretreated cellulosic material converts at least 50% of the cellulose to at least one fermentable sugar. In still a more particular embodiment, hydrolyzing the pretreated cellulosic material converts at least 60% of the cellulose to at least one fermentable sugar. In yet a more particular embodiment, hydrolyzing the pretreated cellulosic material converts at least 70% of the cellulose to at least one fermentable sugar. In still yet a more particular embodiment, hydrolyzing the pretreated cellulosic material converts at least 80% of the cellulose to at least one fermentable sugar. In a more particular embodiment, hydrolyzing the pretreated cellulosic material converts at least 90% of the cellulose to at least one fermentable sugar. In still a more particular embodiment, hydrolyzing the pretreated cellulosic material converts at least 91% of the cellulose to at least one fermentable sugar. In yet a more particular embodiment, hydrolyzing the pretreated cellulosic material converts at least 92% of the cellulose to at least one fermentable sugar. In still yet a more particular embodiment, hydrolyzing the pretreated cellulosic material converts at least 93% of the cellulose to at least one fermentable sugar. In a more particular embodiment, hydrolyzing the pretreated cellulosic material converts at least 94% of the cellulose to at least one fermentable sugar. In still a more particular embodiment, hydrolyzing the pretreated cellulosic material converts at least 95% of the cellulose to at least one fermentable sugar. In yet a more particular embodiment, hydrolyzing the pretreated cellulosic material converts at least 96% of the cellulose to at least one fermentable sugar. In still yet a more particular embodiment, hydrolyzing the pretreated cellulosic material converts at least 97% of the cellulose to at least one fermentable sugar. In a more particular embodiment, hydrolyzing the pretreated cellulosic material converts at least 98% of the cellulose to at least one fermentable sugar. In still yet a more particular embodiment, hydrolyzing the pretreated cellulosic material converts at least 99% of the cellulose to at least one fermentable sugar.

In a more particular embodiment, hydrolyzing the pretreated cellulosic material converts at least 50% of the hemicellulose to at least one fermentable sugar. In still a more particular embodiment, hydrolyzing the pretreated cellulosic material converts at least 60% of the hemicellulose to at least one fermentable sugar. In yet a more particular embodiment, hydrolyzing the pretreated cellulosic material converts at least 70% of the hemicellulose to at least one fermentable sugar. In still yet a more particular embodiment, hydrolyzing the pretreated cellulosic material converts at least 80% of the hemicellulose to at least one fermentable sugar. In a more particular embodiment, hydrolyzing the pretreated cellulosic material converts at least 90% of the hemicellulose to at least one fermentable sugar. In still a more particular embodiment, hydrolyzing the pretreated cellulosic material converts at least 91% of the hemicellulose to at least one fermentable sugar. In yet a more particular embodiment, hydrolyzing the pretreated cellulosic material converts at least 92% of the hemicellulose to at least one fermentable sugar. In still yet a more particular embodiment, hydrolyzing the pretreated cellulosic material converts at least 93% of the hemicellulose to at least one fermentable sugar. In a more particular embodiment, hydrolyzing the pretreated cellulosic material converts at least 94% of the hemicellulose to at least one fermentable sugar. In still a more particular embodiment, hydrolyzing the pretreated cellulosic material converts at least 95% of the hemicellulose to at least one fermentable sugar. In yet a more particular embodiment, hydrolyzing the pretreated cellulosic material converts at least 96% of the hemicellulose to at least one fermentable sugar. In still yet a more particular embodiment, hydrolyzing the pretreated cellulosic material converts at least 97% of the hemicellulose to at least one fermentable sugar. In a more particular embodiment, hydrolyzing the pretreated cellulosic material converts at least 98% of the hemicellulose to at least one fermentable sugar. In still yet a more particular embodiment, hydrolyzing the pretreated cellulosic material converts at least 99% of the hemicellulose to at least one fermentable sugar.

In still yet another embodiment, methods described herein disclose a process for isolating lignin from cellulosic material for enzymatic hydrolysis, comprising:

a. mixing a cellulosic material and at least cellulosic solvent alcohol in a reactor to form a slurry;

b. heating and shearing the slurry at a temperature in the range from equal to or more than 100° C. to equal to or less than 300° C. to obtain a pretreated cellulosic material;

c. extracting lignin from the pretreated cellulosic material, wherein the lignin has a number average molar mass (Mₙ) in the range from equal to or more than 1,000 to equal to or less than 10,000.

Aspects of steps a. through c. can be carried out as described above and the method may or may not further comprise one or more additional steps (e.g., recovery, washing, drying, etc.). In particular aspects the extracted lignin will have a number average molar mass (Mn) of at least 1,000 daltons (e.g. 1,000 daltons, 2,000 daltons, 3,000 daltons, 4,000 daltons, 5,000 daltons, 6,000 daltons, 7,000 daltons, 8,000 daltons, 9,000 daltons 10,000 daltons, and so on). In a particular aspect, the extracted lignin will have a Mn of 5,784 daltons.

In another aspect, the extracted lignin will have a weight average molecular weight (Mw) of at least 19,000 daltons (e.g. 19,000 daltons, 19,100 daltons, 19,200 daltons, 19,300 daltons, 19,400 daltons, 19,500 daltons, 19,600 daltons, 19,700 daltons, 19,750 daltons 19,800 daltons, 19,850 daltons, 19,900 daltons, 19,950 daltons, 20,000 daltons, and so on). For example, the extracted lignin can have a Mw of 19,849 daltons.

In still yet another embodiment, methods described herein disclose a process for isolating xylan from cellulosic material, comprising:

a. mixing a cellulosic material and at least cellulosic solvent alcohol in a reactor to form a slurry;

b. heating and shearing the slurry at a temperature in the range from equal to or more than 100° C. to equal to or less than 300° C. to obtain a pretreated cellulosic material;

c. extracting xylan from the pretreated cellulosic material;

d. separating water insoluble xylan and water soluble xylan from the extracted xylan; and

e. recovering the water insoluble xylan from the extracted xylan, wherein the water insoluble xylan has a number average molar mass (Mₙ) in the range from equal to or more than 30,000 to equal to or less than 60,000.

Aspects of steps a. through c. can be carried out as described above and the method may or may not further comprise one or more additional steps (e.g., recovery, washing, drying, etc.). Aspects of steps c. and d. may be performed through additional stages of washing and separation carried out as described above.

In particular aspects the recovered water insoluble xylan will have a number average molar mass (Mₙ) of at least 30,000 daltons (e.g. 30,000 daltons, 32,500 daltons, 35,000 daltons, 37,500 daltons, 40,000 daltons, 42,500 daltons, 45,000 daltons, 47,500 daltons, 5,000 daltons, 52,500 daltons, 55,000 daltons, 57,500 daltons, 60,000 daltons, and so on).

In a particular aspect, the recovered water insoluble xylan will have a Mₙ of 3,940 daltons. In another particular aspect, the recovered water insoluble xylan will have a Mₙ of 40,600 daltons. In yet another particular aspect, the recovered water insoluble xylan will have a Mₙ of 41,200 daltons. In still yet another particular aspect, the recovered water insoluble xylan will have a Mₙ of 41,400 daltons. In yet still another particular aspect, the recovered water insoluble xylan will have a Mₙ of 43,100 daltons. In another particular aspect, the recovered water insoluble xylan will have a Mₙ of 44,100 daltons. In still another particular aspect, the recovered water insoluble xylan will have a Mₙ of 48,700 daltons. In yet another particular aspect, the recovered water insoluble xylan will have a Mₙ of 52,500 daltons. In still yet another particular aspect, the recovered water insoluble xylan will have a Mₙ of 55,400 daltons.

In another aspect, the recovered water insoluble xylan will have a weight average molecular weight (Mw) of at least 45,000, daltons (e.g. 45,000 daltons, 46,000 daltons, 47,000 daltons, 48,000 daltons, 49,000 daltons, 50,000 daltons, 51,000 daltons, 52,000 daltons, 53,000 daltons, 54,000 daltons 55,000 daltons, 56,000 daltons, 57,000 daltons, 58,000 daltons, 59,000 daltons, 60,000 daltons, 61,000 daltons, 62,000 daltons, 63,000 daltons, 64,000 daltons, 65,000 daltons, 66,000 daltons, 67,000 daltons, 68,000 daltons, 69,000 daltons, 70,000 daltons, and so on).

In a particular aspect, the recovered water insoluble xylan will have a Mw of 45,600 daltons. In another particular aspect, the recovered water insoluble xylan will have a Mw of 45,900 daltons. In yet another particular aspect, the recovered water insoluble xylan will have a Mw of 46,200 daltons. In still yet another particular aspect, the recovered water insoluble xylan will have a Mw of 47,000 daltons. In yet still another particular aspect, the recovered water insoluble xylan will have a Mw of 47,400 daltons. In another particular aspect, the recovered water insoluble xylan will have a Mw of 49,700 daltons. In still another particular aspect, the recovered water insoluble xylan will have a Mw of 55,000 daltons. In yet another particular aspect, the recovered water insoluble xylan will have a Mw of 5,720 daltons. In still yet another particular aspect, the recovered water insoluble xylan will have a Mw of 65,600 daltons. In yet still another particular aspect, the recovered water insoluble xylan will have a Mw of 68,100 daltons.

In another aspect the recovered water insoluble xylan will have a number average degree of polymerization (DPₙ) of at least 80 (e.g., 80, 85, 90, 95, 100, 105, 110, 115, 120, 125, 130, 135, 140, 145, 150, 155, 160, 165, 170, 175, 180, 185, 190, 195, 200, and so on).

In a particular aspect, the recovered water insoluble xylan will have a DPₙ of 105.8. In another particular aspect, the recovered water insoluble xylan will have a DPₙ of 109.0. In still another particular aspect the recovered water insoluble xylan will have a DPₙ of 110.7. In yet another particular aspect, the recovered water insoluble xylan will have a DPₙ of 111.2. In still yet another particular aspect the recovered water insoluble xylan will have a DPₙ of 115.8. In yet still another particular aspect, the recovered water insoluble xylan will have a DPₙ of 118.5. In another particular aspect, the recovered water insoluble xylan will have a DPₙ of 130.9. In still another particular aspect, the recovered water insoluble xylan will have a DPₙ of 141.2. In yet another particular aspect, the recovered water insoluble xylan will have a DPₙ of 149.0.

### EXAMPLES

The following examples are provided for illustrative purposes and are not intended to limit the scope of the invention as claimed herein. Any variations in the exemplified examples which occur to the skilled artisan are intended to fall within the scope of the present invention.

### Examples 1-6

### Glycerol Thermal Pretreatment of Sweet Gum and Corn Stover

Materials:

Biomass:

Mature sweet gum (Liquidambar styraciflua) or corn stover was ground in a knife mill and sieved to size (fine powder <80 mesh) or fiber (40<X<60 mesh). The sweet gum was extracted to produce extractive free wood and both samples were conditioned to 8% moisture content.

Solvents/Reagents:

Glycerol (ACS certified), acetone (grade), and n-dimethylformamide (DMF) (grade) were used as received from Sigma Aldrich.

Enzymes:

A commercially available cellulase (Celluclast® from Novozymes) was used at 15 filter paper units (FPU) loading per gram of cellulose in the lignocellulose material.

Reactor (Mixer)/Melt Compounding Equipment:

A three-piece mixer head (commercially available from C. W. Brabender) was mounted on a batch-style counter-rotating, heated mixing chamber (C. W. Brabender Prep-Mixer) and further equipped with a Prep-Center® drive unit (commercially available from C. W. Brabender) and used to process the materials.

Methods:

Glycerol Thermal Pretreatment (GTP)/Melt compounding

The mixer was preheated (either 200° C. or 240° C.) and the sample (10 g to 15 g) was added to the mixer with the blades rotating at 50 rpm. Immediately residual moisture boiled from the fiber/powder. Glycerol was slowly poured into the sample until a past like consistency was noticed on the blades (15 g to 30 g). The amount of glycerol loading was effected by the surface area of the powder. After 8 minutes of mixing, samples were removed from the mixer. Mass of the samples before and after melt processing was within experimental error of recovering the mass from the melt mixer.

Hydrolysis

Hydrolysis can be performed according to standard conditions known in the art.

Lignin Extraction of Sweet Gum

Treated samples were heated to 60° C. in distilled water and filtered. Samples were washed with acetone and air-dried. The samples were placed into DMF and soaked for 24 hours. The black liquor was filtered and subsequently concentrated under reduced pressure in a rotary evaporator (i.e, a “roto-vap”). An approximately 50 ml sample was slowly poured into 1 L of acidified water (0.1 N HCl) precipitating the lignin. Samples of the lignin were collected by centrifugation and washed 3× with distilled water. The samples were lyophylized and stored for further evaluation.

Lignin Analysis

The molecular weight was analyzed of the lignin acetate derivative via GPC using tetrahydrofuran as the mobile phase. Lignin functionality was analyzed of the phosphitylated derivative using 31P NMR.

### Example 1

### Compositional Analysis of Sweet Gum Subjected to GTP

Glycerol thermal pretreatment (GTP) was performed on Sweet Gum according to the methods described (see the Materials and Methods section above) at 200° C. and 240° C. respectively to heat and shear the sweet gum biomass material. Upon completion of the GTP, the following samples were obtained:

GTP Compounded Sample at 200° C. (“GTP Sample 200° C.”)

GTP Compounded Sample at 240° C. (“GTP Sample 240° C.”) and

Untreated Wood Flour Sample (Control Sample, no heat exposure)

The Control Sample did not receive any pretreatment other than milling (i.e., melt compounding without the presence of glycerol). Samples were further extracted with DMF, washed with acetone and water, and then vacuumed dried.

In duplicate, acid-insoluble Klason analysis and monosaccharide analysis were performed on the GTP samples and control samples, which results are in Table 1.

TABLE 1
Compositional analysis of extracted melt compounded and
control samples.

| Sample | Glucan | Xylan | Galactan | Arabinan | Mannan | Lignin |
|---|---|---|---|---|---|---|
| Reference | 39.4% | 17.5% | 0.8% | 0.3% | 3.1% | 23.7% |
| values¹ | | | | | | |
| Control | 34.8% | 16.6% | 0.2% | 0.7% | 1.9% | 22.0% |
| values | | | | | | |
| Com- | 45.4% | 15.6% | 0.0% | 0.4% | 2.4% | 17.6% |
| pounded | | | | | | |
| at 200° C. | | | | | | |
| Com- | 59.8% | 16.9% | 0.0% | 0.3% | 2.7% | 7.6%² |
| pounded | | | | | | |
| at 240° C. | | | | | | |

¹Reference values are from literature (Johnson, J. (2011)

Cellulosic ethanol production lags, Chemical and Engineering News, October 10, pg. 12). As indicated in Table 1, GTP processing at 200° C. does not significantly impact the remaining lignin and saccharide composition of the extracted material. Table 1 further indicates that significant lignin remained associated with the control sample and that there is limited change in the saccharide composition of the experimental sample when melt compounding at 200° C. relative to the control sample. Table 1 indicates that when samples are GTP processed at the higher temperature of 240° C., a significant amount of lignin was extracted, corresponding to 65% of the original lignin content. At the higher temperature of 240° C. approximately 2/3^{rd} of the lignin was removed, with xylan and mannan contents remaining relatively constant. Lignin was more extractable at the higher processing temperature.

Further illustrated in Table 1, is the impact glycerol had on the hydrolysis rates of the monosaccharides that are more sensitive, such as the galactan and aribinan components. Methyl-β-D-galactopyrinosides have a higher hydrolysis rate relative to the other monosaccharides components within biomass and the galactan content is reduced significantly. As shown in Table 1, there is no galactan present following GTP processing. Additionally, Table 1 illustrates that arabinan content is reduced at least by half from the GTP processing. Aribinofuranose is a 5-member ring and is more readily hydrolysable because of this structure relative to the monosaccharides in the pyranose rings. Finally, Table 1 illustrates the retention the xylan materials suggesting that GTP processes did not generate a great degree of low molecular weight inhibitory compounds.

### Example 2

### Total Glucan Digestibility of GTP Samples

Sweet Gum samples were prepared and extracted as in Example 1. Samples were further hydrolyzed with cellulase enzymes (Celluclast®) and the amount of glucose from saccharification was normalized to the total glucan content providing total glucan digestibility.

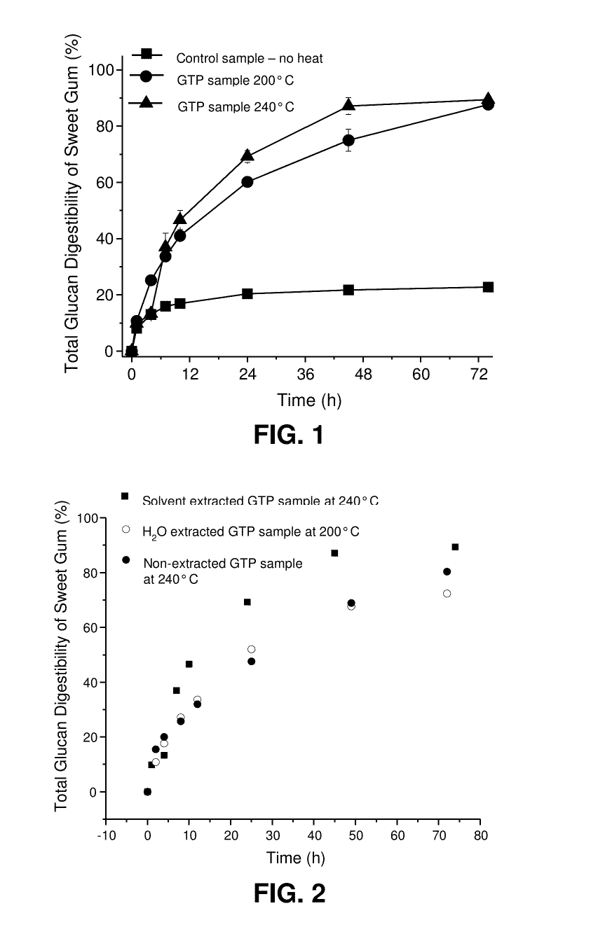

Results are provided in FIG. 1. As shown in FIG. 1, GTP processing significantly changed the degree of recalcitrance of the cellulose as seen by the level of conversion.

For the control sample, only 20% of the cellulose is hydrolyzed into glucose after 72 hrs. Glucose digestability changes significantly for the melt processed biomass after the initial 6 hours where it deviates sharply. Both processing temperatures reveal high glucan digestibility with the 240° C. sample reaching over 85% at 48 hrs, and both 200° C. and 240° C. samples yielding similar glucan digestibility at 72 hrs. It should be noted that the lignin content was significantly greater for the sample at 200° C.; however, this only appears to slightly influence the hydrolysis.

### Example 3

### Effect of Glycerol on Total Glucan Digestibility

The effect of glycerol on total glucan digestibility was investigated because a comparison between the total glucan digestibility of the Sweet Gum GTP samples at various processing temperatures did not show a significant difference in total glucan digestibility (see FIG. 1) despite the significant differences in lignin content between GTP samples compounded at 200° C. and at 240° C. (Table 1).

GTP samples were thermally processed (i.e., compounded) as in Example 1. Following thermal processing, the GTP processed accordingly:

GTP sample compounded at 240° C. and solvent extracted with DMF (“Solvent Extracted GTP Sample at 240° C.”);

GTP sample compounded at 200° C. and extracted with water (“H₂O Extracted GTP Sample at 200° C.”);

GTP sample compounded but not extracted, i.e., without the removal of glycerol. (“Non-extracted GTP sample at 240° C.”).

Following thermal processing, the samples were hydrolyzed directly according to the procedure described in Example 2 and the total glucan digestibility was compared to the total glucan digestibility of the extracted 240° C. sample. Results are provided in FIG. 2.

As illustrated in FIG. 2, the Non-extracted GTP sample at 240° C. shows similar digestibility to the H₂O Extracted GTP Sample at 200° C. During the first 48 hrs., the hydrolysis of the Non-extracted GTP sample at 240° C. and the H₂O Extracted GTP Sample at 200° C. is reduced relative to the Solvent Extracted GTP Sample at 240° C. The data suggests that access to cellulose surfaces may be partially hindered. With the data showing total glucan digestibility still increasing at 48 and 72 hrs. respectively, longer hydrolysis time may yield similar conversion to the solvent extracted materials (e.g., Solvent Extracted GTP Sample at 240° C.).

### Example 4

### Effect of Pretreated Cellulosic Material Particle Size on Total Glucan Digestibility

It was investigated whether a relationship between particle size and the degree of conversion (as measured by total glucan digestibility) existed.

Sweet Gum samples were thermally processed according to the procedures described in Example 1, except the samples were extracted with water instead of DMF. The following samples were obtained:

Fine particle (fine powder <80 mesh) GTP sample compounded at 240° C. and extracted with H₂O (“Water Extracted, Fine Particle Sample”);

Large particle (fibrous 40<X<60 mesh) GTP sample compounded at 240° C. and extracted with H₂O (“H₂O Extracted, Large Particle Sample”);

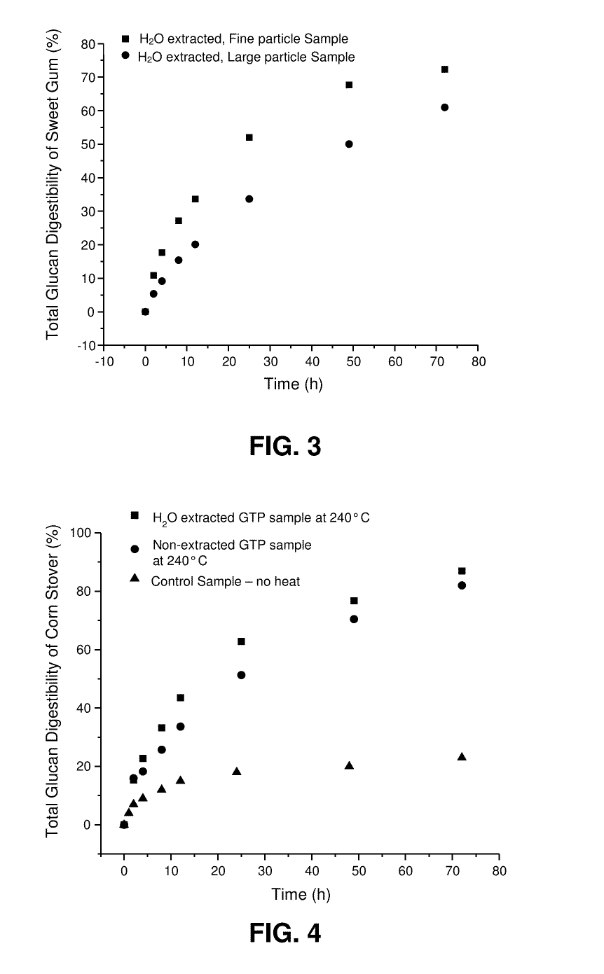

The samples were subsequently hydrolyzed according to the procedures described in Example 2. Results are provided in FIG. 3.

As illustrated in FIG. 3, hydrolysis of the Water Extracted, Fine Particle Sample was much greater than the H₂O Extracted, Large Particle Sample. This suggests that the amount of available surface area dramatically impacts hydrolysis rate. Longer hydrolysis times (or higher enzyme loadings) may be desired for enhanced digestibility.

### Example 5

### Total Glucan Digestibility of Corn Stover

Woody materials currently make up a substantial portion of the biomass available for conversion into fermentable sugar intermediates, which can in turn be processed downstream into other chemical intermediates, such as ethanol. Agricultural residues, such as corn stover, can also be a source of biomass available for conversion.

The total glucan digestibility of corn stover was investigated. Corn stover was thermally processed in the presence of glycerol (GTP processed) according to the methods described (see the Materials and Methods section above) except the samples were extracted with water instead of DMF. The following samples were obtained:

GTP sample compounded at 240° C. and extracted with water (“H₂O Extracted GTP Sample at 240° C.”);

GTP sample compounded but not extracted, i.e., without the removal of glycerol. (“Non-extracted GTP sample at 240° C.”).

Untreated Corn Stover Sample (Control Sample, no heat exposure)

The Control Sample did not receive any pretreatment other than milling (i.e., melt compounding without the presence of glycerol). The Samples were further extracted with water and then subsequently vacuumed dried.

The samples were enzymatically hydrolyzed as described herein (see the Materials and Methods section above) and the total glucan digestibility was determined according to the procedure used in Example 2. Results are in FIG. 4.

As shown in FIG. 4, untreated corn stover has relatively low cellulose hydrolysis to glucose ratio with only 20% of the available glucose reached. In comparison, GTP processed corn stover showed significant increase with glucose digestibility reaching nearly 85%. At 48 and 72 hrs. respectively, the data indicates that total glucan digestibility was still increasing, suggesting that the conversion has not reached a plateau.

The data further suggests that conversion may continue to occur when the duration time of hydrolysis is increased for the non-solvent extracted sample; however, the non-solvent extracted sample still showed significant improvement from control.

### Example 6

### Analysis of GTP Extracted Lignin

Lignin isolated from the melt processed sweet gum was analyzed (see Materials and Methods above) for its functionality and molecular weight, two preliminary key attributes that are important for its utilization.

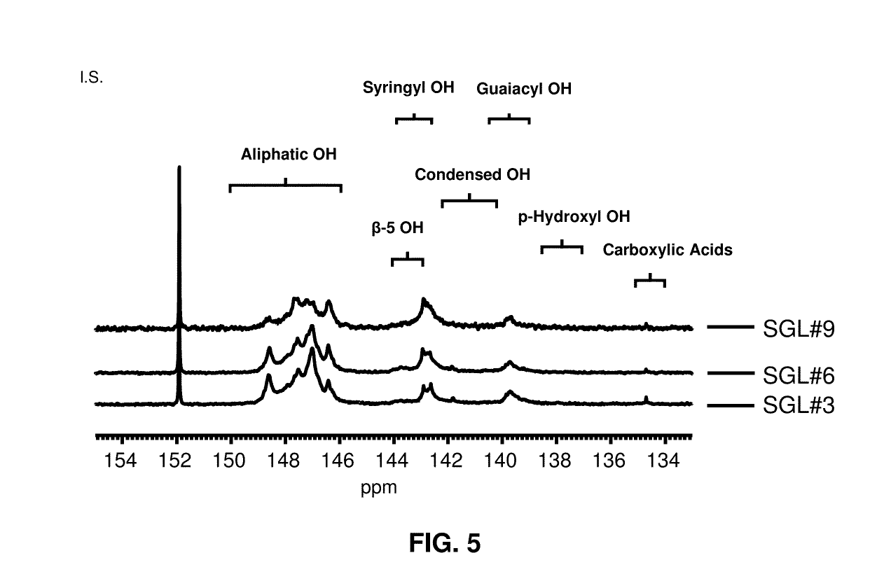

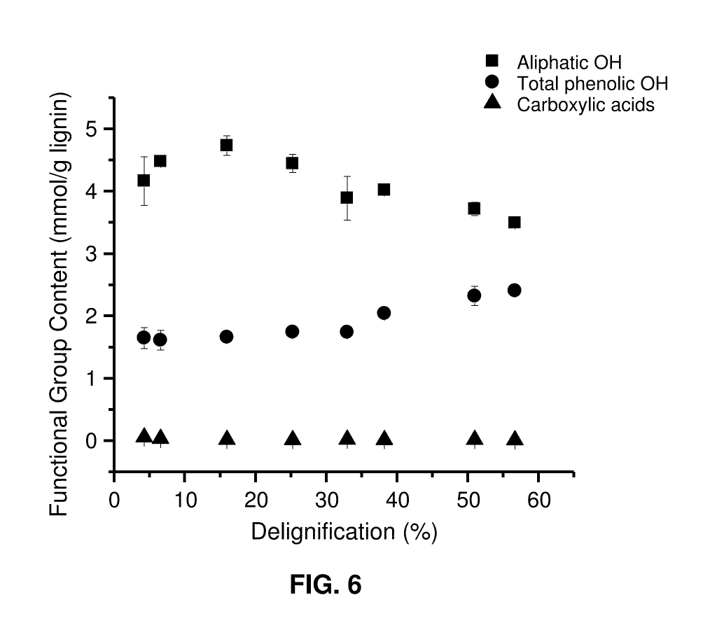

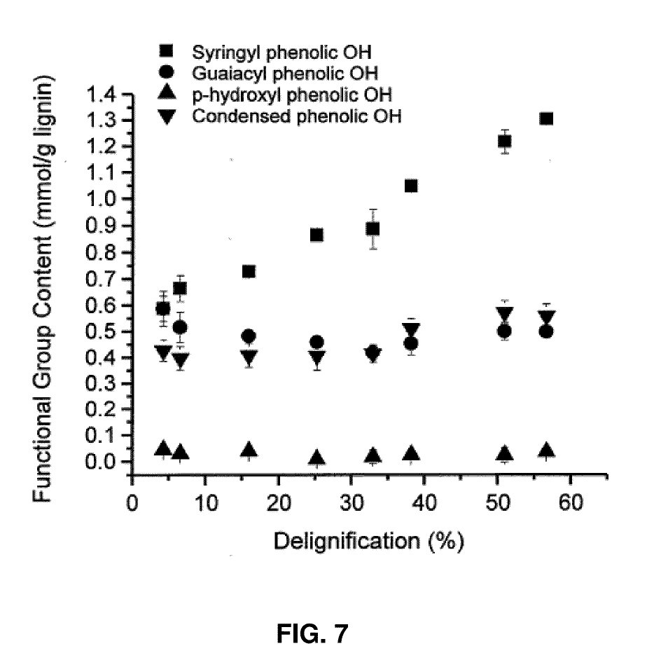

Quantitative 31P NMR analysis provided the concentration of hydroxyl groups and carboxylic acid as shown in FIG. 5. Phenolic hydroxyl content of the syringyl/5-substituted type was 0.47 mmol/g of the lignin compared to 0.07 mmol/g guaiacyl content with an aliphatic hydroxyl content of 1.31 mmol/g. (See FIGS. 6-7).

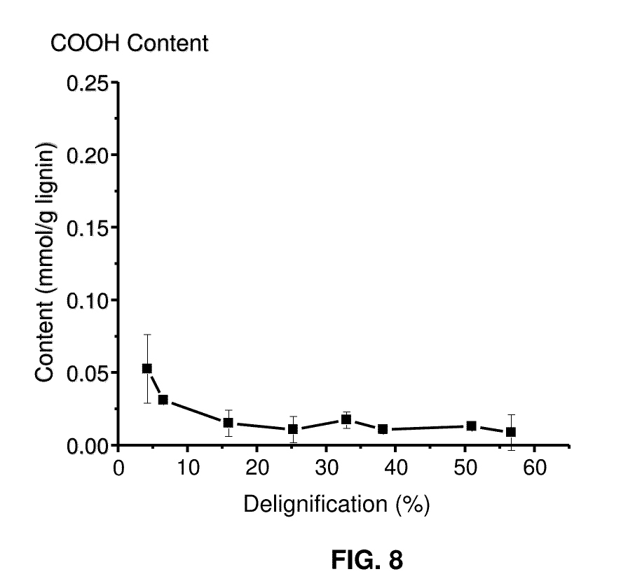

The absence of detectable carboxyl groups revealed that the process does not involve significant oxidation. (See FIG. 8). The sample has a relative low phenolic content, with a high syringyl to guaiacyl ratio, and no carbonyl content. These data suggest a non-condensed, high molecular weight lignin.

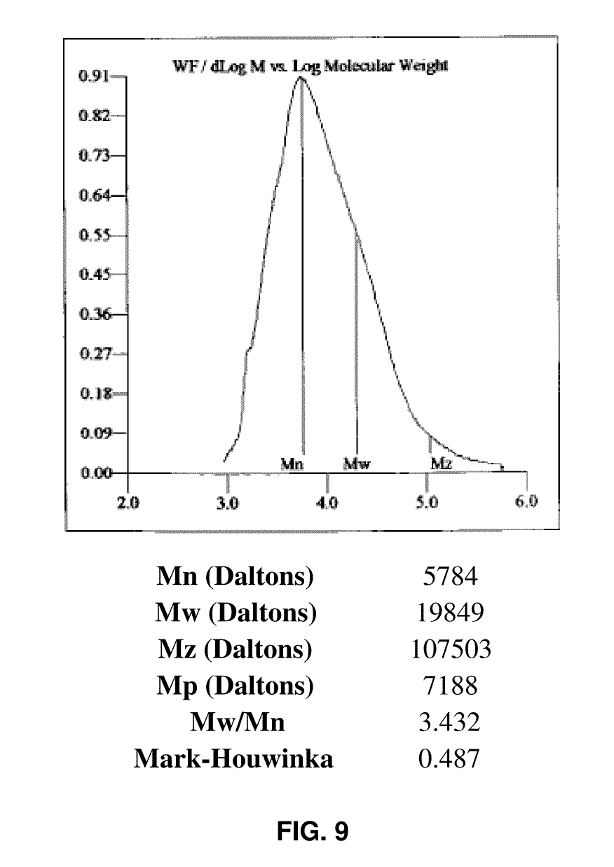

The lignin was derivatized and the molecular weight was analyzed via GPC. As can be seen from the GPC trace, there is a low MW shoulder and a high MW tail as shown in FIG. 9. The number average MW (Mₙ) is 5,784 daltons with a weight average molecular weight (M_{w}) of 19,849 daltons, resulting in a polydispersity index of 3.4. The Mark-Houwink-Sakurada (MHS) exponential parameter is 0.487. (See FIG. 9). These numbers are significant compared to lignin derived from other pretreatments such as organosolv, which have molecular weights much lower, or dilute acid that has highly oxidized and condensed lignin relative to the melt processed lignin.

Compared to other pretreatment/pulping processes the MHS is significantly higher indicating the lignin has properties near a free draining coil in a theta solvent (0.5). The data suggests a high degree of linearity for a technical lignin along with a high molecular weight.

### Examples 7-8

### Glycerol Thermal Pretreatment of Brewers Spent Grain

Materials:

Brewer's Spent Grain (BSG):

Brewer's spent grain was provided by the Highland Brewing Company, Asheville, N.C. on an “as-needed” basis. Several different beer production runs were represented. Gaelic Ale BSG and St. Terese's Pale Ale BSG (product #2697) were the biomass resources for the analysis and process experimentation.

The National Renewable Energy Laboratory standard procedures for biomass were followed for biomass preparation (2008) and determination of carbohydrates and lignin (2011). The BSG had a moisture content of 75.2% (24.8% solids). The dry BSG matter had a crude protein content of 22.8%, a cellulose content of 17.2%, hetero-polysaccharide content of 16.7% (water-insoluble) acid insoluble lignin content of 9.0%, ash content of 3.2%, and extractives content including lipids and water soluble polysaccharides of the remaining material (31.1%).

Solvents/Reagents:

Glycerol (Sigma-Aldrich #536407 ACS reagent 99.5%)

Reactor (Mixer)/Melt Compounding Equipment:

A CW Brabender counter rotating twin-screw extruder (CTSE), model-V, with high intensity mixing shear screws was installed on a Brabender Prep-Center; model VD-52, with heating and cooling systems controlled by a Brabender Temperature Control Center; No. 2301. The system has been plumbed with cooling lines (air and water) for temperature control and suitable electric service has been supplied to the system. The continuous high shear mixing system functionality was further enhanced with an appropriate vapor removal system.

Three additional modifications were made to the Brabender CTSE equipment in an effort to adapt the extruder to more uniform and predictable biomass processing with glycerol. The first was the addition of a weighted pushrod to maintain a constant pressure on the material in the feed opening of the CTSE. The weight is 3,226 g and has a pushrod end diameter about 2 mm smaller than the diameter of the feed opening. It is supported by rope and pulleys for easy removal when it is time to add more material.

The second modification was the removal of the fourth stage of heating and compression from the outlet end of the C W Brabender CTSE. Brabender calls this the “collector head” and the “collector insert.” Both of these pieces of hardware were designed to collect and compress material in the twin screw chamber into a single, centerline symmetrical, outlet port approximately 18 mm in diameter. The hardware was removed to allow for GTP processing.

The third modification incorporated a stainless steel tubing adaptor that was found to almost match the diameter of the CTSE outlet port with the collector insert removed. The adaptor was fitted with brass shim stock until the diameters had a snug fit when put together. This retrofit enabled outlet from the lowest portion of the twin screw chamber and no upward motion, plus it did not force any compression of material exiting the twin screw chamber.

The system was designed for conveying, mixing, and extruding homogeneous, uniform, hydrocarbon polymer pellets and not heterogeneous, non-uniform biomass particles. The screw shafts measured 30 mm diameter at inlet, 19 mm at outlet, with an effective screw length of approximately 310 mm from the beginning of the inlet feed area to the tip of the screws.

Centrifuge:

A Bock basket centrifuge with a 16 kg dry matter maximum, 1750 RPM speed, a maximum single cycle of ten minutes, and either 20 μm or 60 μm filter bags to fit in the basket was used. Bock was acquired by North Star Engineered Products and the most similar current model is the North Star model 215.

Methods:

Extruder/Melt Compounding Preparation:

Experimentation with the counter-rotating twin screw extruder system included various RPM speed settings and temperature settings. Barrel temperatures were variable across three zones (after the fourth heat zone had been removed to modify the outlet). However, as the barrel was a single continuous piece of steel each heat zone was affected by the adjacent one and in the interest of repeatability all zones were always set to identical temperatures.

Three temperatures were used to prepare different sample material: 200° C., 210° C., and 220° C. Two screw speeds were used: 15 RPM and 20 RPM. Various markers were attempted while timing the throughput, estimating residence time by measuring movement of a marker on the exposed twin screws in operation; none of the techniques attempted were effective. The measurement of time when biomass was first introduced into an empty and clean chamber and then when it first exited the system resulted in one estimate. This estimate was affected by the amount of time required to fill the empty chamber. A second estimate was also used when the last material went into a full chamber until the last material appeared to exit the chamber. This estimate was hampered by a very unclear endpoint as material could continue to exit in smaller and smaller amounts as the chamber emptied.

BSG Process Overview:

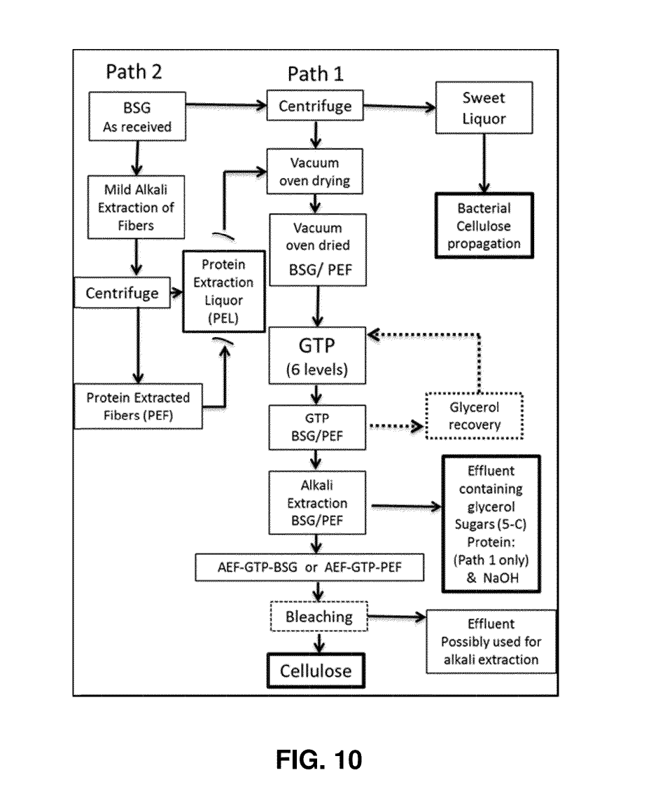

A literature review regarding protein isolation from grain products and BSG in particular (Celus, I. et al 2009; Ervin, V. et al 1989; Connolly, A. 2013; Swanson, B. 1990, Diptee, R., 1989) indicated that a large percentage of the protein components of BSG could be solubilized in a mild, aqueous sodium hydroxide solution at moderate temperature over a period many hours. This step would be conducted in the method prior to drying and blending with glycerol for the GTP. As an initial step toward the total fractionation of the AR-BSG, efficient removal of the residual “sweet liquor” (“sweet liquor”, as used herein, means the residual liquid on the grains after the worth has been removed). The combined result of both of these paths resulted in a simplified 2-path fractionation scheme for BSG processing (see, FIG. 10).

The process flow diagram (PFD) shown in FIG. 10. was the method used for processing BSG and represents a repeatable sequence of process steps that produced high purity cellulose at kilogram scale across several different, intentional, process variable changes.

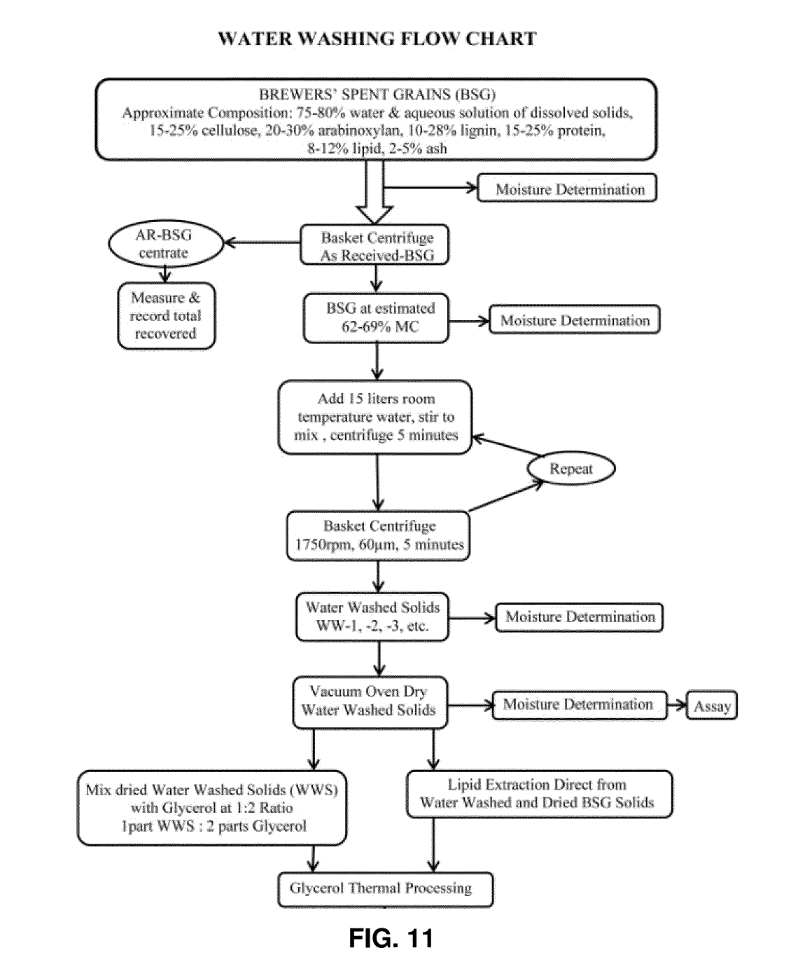

As shown in FIG. 10, there are two paths (i.e., “Path 1” and “Path 2”) and each begin with “as-received Brewer's Spent Grain (AR-BSG) from Highland Brewing Company. Washing procedures are described in FIG. 11.

Path 1 has the option to remove the sweet liquor, dry the material, and then processes it in the twin-screw extruder in the presence of glycerol (GTP processing) prior to extraction and purification. Samples were washed in 5 batches, vacuum dried and subsequently mixed with glycerol at a 1:2 ratio (solids to liquids) and left to soak overnight. The soaking period enables increased uniformity in the distribution and wetting of the dried BSG with glycerol. The 1:2, BSG: glycerol, blended sample was continuously processed at a rate of 369 g/hour on the Brabender conical twin screw extractor set to a temperature of 200° C., operating at 15 rpm. The corresponding residence time for this material was approximately 3 min, providing an overall low severity processing condition.

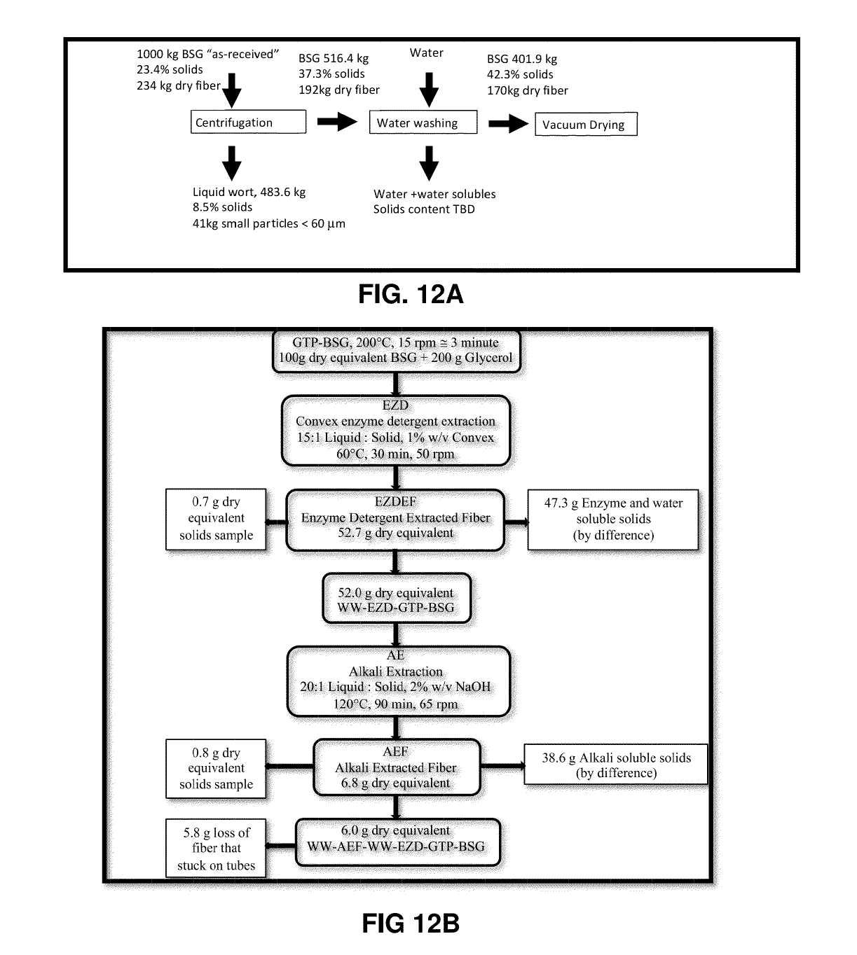

FIG. 12A is a flow diagram illustrating the mass balance for the water washing and drying steps. The data was scaled to 1 metric ton condition wet BSG basis. As can been seen in FIG. 12A, there is significant mass extracted in the process as the total solids after centrifugation and washing is 72% of the initial dry solids matter. The approximate 28% of the initial dry material is suggested to be useful as a nutrient growth broth. At the process temperature of 200° C., there was no mass loss during the glycerol thermal processing (GTP) step. The only difference in mass was attributed to moisture loss of the vacuum dried biomass. After GTP step the samples underwent two different extraction methods.

Extraction may be performed following GTP processing and in the presence of an enzymatic detergent (see FIG. 12B). In one method the samples were extracted with a detergent to remove water-soluble material and glycerol. The detergent had an active enzyme to help remove residual protein and lipids within the BSG. A 1% solution of the detergent was used in this procedure (referred to as ‘EZD’). After detergent extraction, the BSG was extracted with alkali. FIG. 12B illustrates that 1) the total solids content is decreased by 47% of original dry BSG due to the EZD step, indicating significant removal of soluble biomass, and 2) the alkali extraction is almost equally effective removing an additional 39% of original dry BSG, providing an overall low yield of fiber, 6% of original dry BSG.

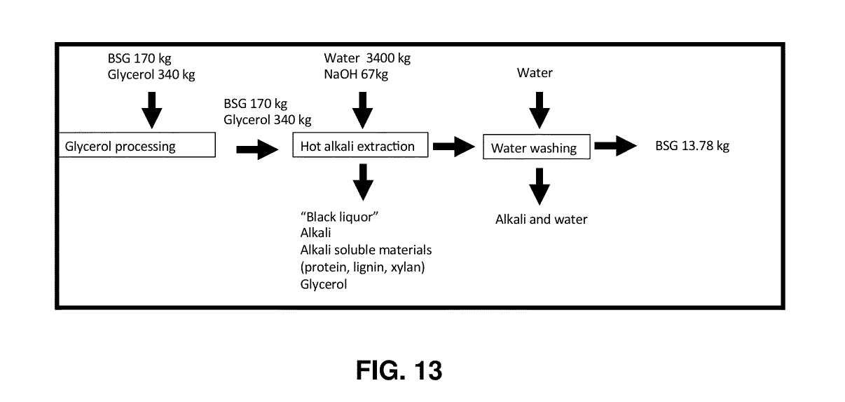

Path 2 places the wet BSG into a mild alkali extraction capturing the weak-alkali soluble components from the BSG prior to GTP in order to minimize heat exposure to the protein located within the BSG (see FIG. 13). As illustrated in FIG. 13, path 2 avoided the enzyme extraction step and glycerol washing. The data is presented in the scaled version to provide an idea of the sodium hydroxide requirement for every ton of wet biomass that has been water extracted.

Bleaching and Chemical Composition of BSG

After alkali extraction, a three stage bleaching treatment was performed on all samples (including the AEF-EZD samples) as reported according to (Mussatto et al. 2008) in order to obtain high purity cellulose. The process used hydrogen peroxide and sodium hydroxide in the first two stages, followed by a sodium hydroxide final stage. After each stage the samples were washed to neutral pH and centrifuged to approximately 19% solids content prior to the subsequent stage. The sample after each bleaching stage was analyzed for their chemical composition. Significant increase in cellulose was found for these samples with xylan as the second most abundant material. Results are in Tables 2 and 3.

TABLE 2
Chemical Composition of BSG that underwent enzyme detergent and alkali
extraction before bleaching occurred, at various levels of bleaching

| | anhydro | | anhydro | | anhydro | | anhydro | | anhydro | |
|---|---|---|---|---|---|---|---|---|---|---|
| | ARAB % | STD % | GALA % | STD % | GLU % | STD % | XYL % | STD % | MAN % | STD % |
| S1 | 1.19 | 0.07 | 0.26 | 0.002 | 70.01 | 2.62 | 14.43 | 0.44 | 0.99 | 0.13 |
| S2 | 0.94 | 0.003 | 0.17 | 0.002 | 70.17 | 0.68 | 12.48 | 0.03 | 0.76 | 0.01 |
| S3V | 0.84 | 0.005 | 0.17 | 0.004 | 80.67 | 0.86 | 11.83 | 0.07 | 0.84 | 0.01 |
| S3O | 0.81 | 0.02 | 0.17 | 0.002 | 76.32 | 1.39 | 11.41 | 0.35 | 0.79 | 0.001 |

S1 = stage 1 fibers, oven dried;
S2 = stage 2 fibers, oven dried;
S3V = stage 3 fibers, vacuum oven dried;
S3O = stage 3 fibers, oven dried.
Drying method refers to final drying step prior to chemical composition analysis.

TABLE 3
Lignin Composition of BSG that underwent enzyme
detergent and alkali extraction before bleaching
occurred, at various levels of bleaching

| | Klason | |
|---|---|---|
| | lignin % | STD % |
| S1 | 4.99 | 2.48 |
| S2 | 4.82 | 0.18 |
| S3V | 1.92 | 0.06 |
| S3O | 3.95 | 0.90 |

S1 = stage 1 fibers, oven dried; S2 = stage 2 fibers, oven dried; S3V = stage 3 fibers, vacuum oven dried; S3O = stage 3 fibers, oven dried. Drying method refers to final drying step prior to chemical composition analysis.

It should be noted in Table 2 that cellulose purity increases with the third stage compared to the first 2 stages. This data suggests that multiple stage bleaching reduces lignin content on the fiber (see Table 3). It should be further noted that the drying method impacted the analysis. When the samples were vacuumed dried at lower temperature, the cellulose content was the highest. This may be attributed to oven drying at much higher temperature causing coalescence of the fiber, preventing complete analysis of the cellulose components. As shown in the Klason lignin analysis (See Table 3) the Klason lignin value appears artificially large, because it is only based on a gravimetric measurement. It is believed the anhydroglucan values would be even greater at each stage of the bleaching process if the samples are dried under low temperature vacuum oven conditions for the compositional analysis.

### Example 7

### Preparation of AR-BSG Samples for GTP Processing

Six 20 liter plastic buckets of St. Terese's Ale, lot #2697, were received and placed into cold storage. The average moisture content of the as-received St. Terese's lot #2697 BSG (“AR-BSG”) was determined to be 76.7%. Excessive aqueous liquid with the BSG when blended with glycerol for the glycerol thermal processing may adversely affect the physical processing ability or the chemistry, which occurs during the thermal process.

As an initial step toward the total fractionation of the as-received AR-BSG, removal of additional wort was performed. Several buckets of St. Terese's AR-BGS were treated similarly although the initial mass per bucket was not the same for each sample. The liquid separation was expedited via basket centrifuge at 1,750 rpm, through a 60 μm fabric basket liner, for five minutes duration. Previous centrifuge trials at four minutes and 10 minutes resulted in less than one percentage point difference in changes in solids content and 10 minutes was a long centrifuge process time, therefore a five-minute time was adopted as a reasonable standard procedure for the experimental process.

For a typical bucket of BSG, from an initial AR-BSG wet mass of 10,978 g at 76.6% moisture content (MC), 5,669 g of wet BSG was retained in the centrifuge bag at 62.7% MC. The wet mass loss from the AR-BSG was 5,309 g and the liquid recovered measured to 5.2 liters volume.

The initial centrifuge step recovered 48% (by weight of initial mass) of residual wort, which was then extracted. The recovered liquid was cloudy with particulates. Additional water washing was performed with 15 liters of room temperature water, stirred into the centrifuge basket to mix with BSG, and samples of BSG were collected at each stage for moisture determination. The water wash extract was removed as described above (see FIG. 11) and retained for subsequent solids analysis. The water wash cycle was repeated five times in this example and the results of typical room temperature water-washing of St. Terese's BSG are in Table 4.

TABLE 4
Results of Room Temperature Water Washing of St. Terese's Brewer's
Spent Grains

| | | | | | | | | | | Dry | % of |
|---|---|---|---|---|---|---|---|---|---|---|---|
| | | | | | | | | | | Mass | Dry |
| Status | Initial² | | | Initial | Final | MC | | | Final | loss | Mass |
| of | Wet | | | Dry | Wet | sample | | | Dry | per | loss |
| BSG | Mass | Initial | Initial | Mass | Mass | Wet | Final | Final | Mass | stage | per |
| Solids | (g) | MC % | Solids % | (g) | (g) | (g) | MC % | Solids % | (g) | (g) | stage |
| AR | 10,978 | 76.6 | 23.4 | 2,569 | 5,669 | 7 | 62.7 | 37.3 | 2,115 | 454 | 17.7 |
| WW1 | 5,662 | 62.7 | 37.3 | 2,112 | 4,994 | 6 | 62.2 | 37.8 | 1,888 | 224 | 10.6 |
| WW2 | 4,988 | 62.2 | 37.8 | 1,885 | 4,676 | 8 | 60.1 | 39.9 | 1,866 | 20 | 1 |
| WW3 | 4,668 | 60.1 | 39.9 | 1,863 | 4,594 | 7 | 60.7 | 39.3 | 1,805 | 57 | 3.1 |
| WW4³ | 4,587 | 60.7 | 39.3 | 1,803 | 4,486 | 7 | 58.1 | 41.9 | 1,880 | −772 | −4.32 |
| WW5 | 4,479 | 58.1 | 41.9 | 1,877 | 4,412 | 8 | 57.7 | 42.3 | 1,866 | 10 | 0.6 |

²After row 1-AR, the initial wet mass does not include the mass of the MC sample removed following centrifugation.
³WW4 apparently reflects an anomaly in processing and errant material had become included in the mass determination.

### Example 8

### Fractionation/GTP Processing of BSG

Fractionation of BSG was accomplished with a sequence of equipment pieces. Initially experimentation was performed to help define which process equipment would be most suitable for the intended sequence of controlled and comparable fractionation work across a range of GTP conditions.

Pilot scale processing utilizing the Brabender Conical Twin Screw Extruder (CTSE) was performed on water washed (see Materials and Methods section above) St. Terese's and on ‘as received’ St. Terese's and Gaelic Ale BSG at varying levels of dryness.

Different techniques were used to remove excess water and water soluble solids, to varying levels of dryness as described. In some cases the BSG was left as received and partially dried in a rotation evaporator, air-dried, freeze dried, or vacuum-oven dried. In other cases water washing was employed. (See Materials and Methods section above).

Centrifugation was used for removal of water soluble solids from the BSG. Water washed BSG was either left in the centrifuged state at 63.5% MC, or vacuum-oven dried. The bulk of the St. Terese's BSG was vacuum-oven dried at 25-30 Torr and 45-55° C. to 3.69% MC. Gaelic BSG had not been water washed with the exception of an experimental recirculating boiling water extraction. Gaelic BSG had been air dried as received to 12.2% MC and freeze dried to 7.14% MC.

The following samples were obtained and provided in Table 5 (includes sample type and preparation prior to GTP, or analysis, or both):

TABLE 5
Sample Identification

| Sample | |
|---|---|
| ID No. | Sample Details of Brewer's Spent Grain Treatments |
| 1 | St. Terese's Pale Ale BSG (STPA-BSG), centrifuged and wort collected, |
| | water washed 6x15 L, vacuum oven dried to 3.69% MC, GTP- CTSE 200° |
| | C. @15 rpm, 2.08:1 G:B ratio⁴. |
| | No glycerol removed prior to alkali extraction. |
| 2 | Gaelic Ale BSG (GA-BSG) as received, freeze dried to 7.14% MC, GTP- |
| | CTSE 215° C. @ 6 rpm, 2.15:1 G:B ratio. |
| | No glycerol removed prior to alkali extraction. |
| 3 | St. Terese's, centrifuged and wort collected, water washed 6x15 L, vacuum |
| | oven dried to 3.69% MC, GTP- CTSE 215° C. @18 rpm, 2.08:1 G:B ratio. |
| | No glycerol removed prior to alkali extraction. |
| 4 | STPA-BSG, centrifuged and wort collected, water washed 6x15 L, |
| | centrifuged to 63.5% MC, GTP- CTSE 215° C. @6 rpm, 2.76:1 G:B ratio. |
| | No glycerol/water removed prior to alkali extraction. |
| 5 | STPA-BSG received and rotation evaporated to 51.9% MC, GTP- CTSE |
| | 215° C. @6 rpm, 2.70:1 G:B ratio. |
| | No glycerol/water removed prior to alkali extraction. |
| 6 | STPA-BSG, centrifuged and wort collected, water washed 6x15 L, vacuum |
| | oven dried to 3.69% MC, GTP- CTSE 215° C. @24 rpm, 2.08:1 G:B ratio. |
| | No glycerol removed prior to alkali extraction. |
| 7 | GA-BSG as received, air dried to 12.2% MC, Wiley milled w/1 mm screen, |
| | sieved 40 mesh prior to alkali extraction. |
| 8 | STPA-BSG, centrifuged and wort collected, water washed 6x15 L, vacuum |
| | oven dried to 3.69% MC prior to alkali extraction. |
| 9 | GA-BSG as received, freeze dried to 7.14% MC, GTP- CTSE 215° C. @ 6 |
| | rpm, 2.15:1 G:B ratio. |
| | Water washed until free of glycerol and freeze dried again prior to alkali |
| | extraction. |
| 10 | GA-BSG as received, freeze dried to 7.14% MC prior to alkali extraction. |
| 11 | STPA-BSG as received, vacuum oven dried to 3.69% MC prior to alkali |
| | extraction. |
| 12 | STPA-BSG, centrifuged and wort collected, water washed 6x15 L, |
| | centrifuged to 63.5% MC, GTP- CTSE 215° C. @6 rpm, 2.76:1 G:B ratio. |
| | Water washed until free of glycerol and freeze dried prior to alkali |
| | extraction. |
| 13 | STPA-BSG centrifuged and wort collected, water washed 6x15 L, |
| | centrifuged and rotation evaporated to 51.9% MC, GTP- CTSE 215° C. |
| | @6 rpm, 2.70:1 G:B ratio. |
| | Water washed until free of glycerol and freeze dried prior to alkali |
| | extraction. |
| 14 | GA-BSG as received, air dried to 12.2% MC, milled w/1 mm screen, |
| | sieved 40 mesh, GTP-Batch 240° C., 12 min @ 100 rpm, 2.28:1 G:B ratio. |
| | Water washed until free of glycerol and freeze dried prior to alkali |
| | extraction. |
| 15 | STPA-BSG as received, vacuum oven dried to 3.33% MC. |
| | Not alkali extracted. |
| 16 | STPA-BSG, centrifuged and wort collected, water washed 6x15 L, vacuum |
| | oven dried to 3.69% MC. |
| | Not alkali extracted. |
| 17 | STPA-BSG, centrifuged and wort collected, water washed 6x15 L, |
| | centrifuged to 63.5% MC, GTP- CTSE 215° C. @6 rpm, 2.76:1 G:B ratio. |
| | Water washed until free of glycerol and freeze dried. Not alkali extracted. |

⁴Table 4 includes a G:B ratio, of glycerol to brewer's spent grain ratio glycerol to brewer's spent grain ratio. Due to the experimentation at varying levels of residual moisture in the BSG, all G:B ratios are calculated based on equivalent the dry matter of BSG. Some of these conditions did not result in usable material such as the GTP extrusion with as received BSG (high moisture issues)

Samples were also analyzed for their extractability with alkali 1.0 N NaOH, 50° C. for 60 min (see Table 6). Also listed in Table 6 is the acid insoluble material after compositional analysis. Some GTP BSG samples were washed with water and dried prior to alkali extraction and other samples were directly extracted with alkali (glycerol+alkali) to limit processing steps.

TABLE 6
Alkali extraction data of fiber

| | | | Percent Yield | % Acid Insoluble |
|---|---|---|---|---|
| Sample ID | Initial Dry | Final Dry | (% remaining | Residue (of final |
| No. | Weight (g) | Weight (mg) | solids) | dry weight) |
| 1 | 1.5 | 421.6 | 28.11 | 23.9 |
| 2 | 1.5 | 271.4 | 18.09 | 33.1 |
| 3 | 1.5 | 285.4 | 19.03 | 25.75 |
| 4 | 1.5 | 695.5 | 46.37 | 40.39 |
| 5 | 1.5 | 647.9 | 43.19 | ND |
| 6 | 1.5 | 331.8 | 22.12 | ND |
| 7* | 1.5 | 324.1 | 21.61 | 24.82 |
| 8* | 1.5 | 417.3 | 27.82 | 20.75 |
| 9 | 1.5 | 598 | 39.87 | 35.75 |
| 10 | 1.5 | 333.9 | 22.26 | 25.94 |
| 11* | 1.5 | 366.9 | 24.46 | 26.6 |
| 12 | 1.5 | 559.1 | 37.27 | 33.95 |
| 13 | 1.5 | 612.1 | 40.81 | ND |
| 14 | 1.18 | 345 | 29.24 | 25.89 |

*Samples are control samples that did not undergo GTP.

As shown in Table 6, varying amounts of solid material was removed from the alkali extraction process as the percent remaining solids of biomass ranged from 18% to 46% (see Table 6). The highest residual numbers were from samples that were at higher moisture content, also confirming that moisture impacts GTP of biomass.

Polysaccharide content of the alkali extracted samples are reported in Table 7 and organized in decreasing levels of glucan content. Glucan content relates to the cellulose content of the fiber, although unextracted “as-received” samples can cause overestimation of cellulose content in the fiber because of starch residues.

TABLE 7
Compositional analysis of residue after extraction⁵

| | | | | | | Equivalent kg |
|---|---|---|---|---|---|---|
| | | | | | | of glucan of |
| | | | | | | alkali |
| | | | | | | extracted |
| | | | | | | fiber per 1 |
| Sample | | | | | | ton of initial |
| ID No. | Arabinan % | Xylan % | Mannan % | Galactan % | Glucan % | fiber* |
| 14 | 0.22 | 4.63 | 1.95 | 0.23 | 59.41 | 107 |
| 3 | 0.88 | 9.98 | 1.83 | 0.46 | 57.53 | 109 |
| 1 | 1.03 | 10.58 | 1.6 | 0.44 | 54.17 | 152 |
| 9 | 0.39 | 6.01 | 1.57 | 0.34 | 50.82 | 203 |
| 11** | 10.59 | 19.29 | 1.39 | 1.2 | 48.35 | 245 |
| 2 | 0.38 | 5.64 | 1.8 | 0.38 | 47.09 | 85 |
| 12 | 1.02 | 8.1 | 1.4 | 0.4 | 46.27 | 172 |
| 8** | 8.33 | 18.17 | 1.05 | 0.88 | 45.72 | 279 |
| 4 | 1.09 | 8.68 | 1.25 | 0.39 | 44.14 | 205 |
| 7** | 9.06 | 15.29 | 1.37 | 1.15 | 33.98 | 110 |
| 17 | 3.63 | 16.93 | 0.67 | 0.54 | 27.22 | ND |
| 16 | 6.97 | 15.7 | 0.4 | 0.91 | 24.32 | ND |
| 15 | 6.52 | 14.27 | 0.53 | 0.82 | 24.22 | ND |

*Initial dry fiber mass is either “as received” fiber mass or equivalent dry fiber mass after GTP, before extraction. Data is used from mass balance in Table 5.
**Higher values for control fiber may relate to the presence of residual starch in fiber.
⁵Table note: All values reported on a dry weight basis and are the average of a duplicate analysis. The correction for acetate is not included. None of the samples were extractive free and some had undergone additional processing prior to analysis (ie. the alkali extracted samples) so neither of the terms “% ext. free” and “% as received” fit this data. See sections 5.2 and 5.6 in the NREL LAP, “Determination of Structural Carbohydrates and Lignin in Biomass”.

For the GTP-CTSE samples, the highest purity fiber is from water extracted fiber that was vacuum-oven dried prior to GTP and based on the purity of the extracted fiber and equivalent dry weight after extraction total “glucan” yield is reported (see Table 7). For select processing conditions cellulose content of the fiber is over 50% of the mass with the remaining material composed of xylan and acid insoluble residue.

As shown in Table 7, the results indicate that the glycerol thermal processing causes a significant amount of aribinan and galactan to be removed from the fiber, with a reduction in xylan. In the native state, arabinan is linked to xylan, and the process appears to remove significant amount of arabinan suggesting a more accessible cellulose surface for bleaching. Overall, yields between 10 to 20% of cellulose are possible prior to the bleaching steps. This data would be equivalent to 100 kg to 200 kg of cellulose per ton of dried melt compounded fiber.

### Example 12

### Glycerol Thermal Pretreatment of Tobacco Stems/Midribs

Materials and Methods:

Materials:

Tobacco Midribs/Stems

Tobacco midribs (TMR), or stems, were received from Universal Leaf Corporation. The TMR were sections of stem approximately 50-150 mm in length and 4-8 mm in diameter. The midribs were reported by Universal Leaf to contain approximately 15% cellulose. This value is in disagreement with published literature values which indicates for flue-cured tobacco stems, the cellulose content range was 34-42% (Agrupis S C, Maekawa E, 1999, Industrial utilization of tobacco stalks (I) preliminary evaluation for biomass resources. Holzforschung 53 (1999) p. 29-32; and Agrupis S C, Maekawa E, Suziki K, 2000, Industrial utilization of tobacco stalks (II): preparation and characterization of tobacco pulp by steam explosion pulping. J. Wood Sci (2000) 46:222-229). Another source claimed 41% cellulose (Shen G, Tao H, Zhao M, Yang B, Wen D, Yuan Q, Rao G, 2009, Effect of hydrogen peroxide pretreatment on the enzymatic hydrolysis of cellulose. J. Food Process Eng 34 (2011) 905-921 © 2009 Wiley Periodicals, Inc.).

Reactor (Mixer)/Melt Compounding Equipment:

A CW Brabender counter rotating twin-screw extruder (CTSE) as modified and discussed in Examples 7-8 was used.

Methods:

TMR Preparation

The flue-cured tobacco stems were processed through a Wiley size 4 knife mill fit with an outlet screen having 6 mm diameter holes producing a visibly large percentage of fine particulate material. A brief test was performed with no outlet screen at all and an unacceptable number of oversized (relative to the Brabender conical twin screw extruder (CTSE) capabilities) particles were the result.

The 6 mm screen was put back in place and the remainder of the tobacco material was milled. A solids determination on the milled material was performed in triplicate. The milled material was divided in half by weight for preliminary experimentation and secondary experimentation beginning with the glycerol thermal processing (GTP) step.

### Example 12

### GTP of TMR

Using a 1 mm hand sieve and horizontal shaking action, sub-one millimeter particles (e.g., fines) were removed from the milled TMR sample. This step removed 32% by weight of fines, leaving 1,476 g (89.9% solids) of acceptable TMR. The ˜1.5 kg sample was split in half to reserve material for additional testing as necessary. This material was then mixed with glycerol. 738 g at 89.9% solids of milled and sieved TMR was equivalent to 663 g ODeq.

The batch of TMR at >1 mm mean diameter was mixed with glycerol at 1.8:1 glycerol:TMR (“G:TMR”) and run through the CTSE at 200° C., 20 rpm, and utilizing the 3.2 kg pushrod weight. This material processed at an overall rate of approximately 125 g/h on an oven dry TMR basis. The rate is comparable to the lower end of the range of feed rates with BSG in the same equipment.

The batch GTP processed TMR was extracted following Agrupis et. al. (2000). Two successive extractions of tobacco stalks with 2% sodium hydroxide w/w on oven dry biomass, 90° C., one hour.

The extraction ratio was set to 1:18 fiber to liquor. A deviation to the method was made to accommodate for the slightly acidic filtered tap water used. Two percent (w/w) NaOH loading on ODeq TMR was mixed with deionized water at 1:18 fiber to liquid (10,584 g H₂O), and corresponds to a pH of about 12.4. As mixed with filtered tap water according to Agrupis, et al. (2000), the pH was 12.0 and to attain a pH of 12.4, additional sodium hydroxide pellets were weighed and added to the solution. As pH 12.4 conditions were attained, the actual loading was 4.8% sodium hydroxide on oven dry fibers.

Extraction liquor and solids was vacuum filtered for separation in the range of 20-95 Torr. As 95-100 Torr is registered on the vacuum gauge, the filter cake was removed from the 35 μm filter screen on the 200 mm diameter Büchner funnel. Additional filter cakes were produced in a similar manner.

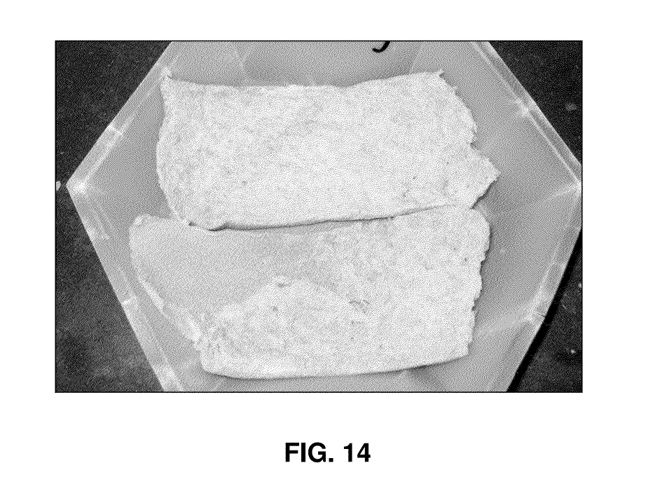

The sieved TMR material performed similar to all dried BSG material in the extruder for the GTP process (see FIG. 14). The data for GTP TMR batch was an 89% closure on the combined glycerol and TMR mass balance. It was assumed that all water in the biomass evaporates at 200° C. during GTP and is not included in the mass balance. Of the 11% loss (not including water loss) it is indeterminate what amounts were from TMR and glycerol.

It will be understood that the Specification and Examples are illustrative of the present embodiments and that other embodiments within the spirit and scope of the claimed embodiments will suggest themselves to those skilled in the art. Although this disclosure has been described in connection with specific forms and embodiments thereof, it would be appreciated that various modifications other than those discussed above may be resorted to without departing from the spirit or scope of the embodiments as defined in the appended claims. For example, equivalents may be substituted for those specifically described, and in certain cases, particular applications of steps may be reversed or interposed all without departing from the spirit or scope for the disclosed embodiments as described in the appended claims. Additionally, one skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A method of processing lignocellulosic biomass, comprising:
providing lignocellulosic biomass and at least one solvent;
providing and heating a mixer to a temperature of between 100° C. to 300° C.;
adding the biomass and the solvent to the mixer;
mixing the biomass and solvent into a biomass slurry; and
melt compounding the biomass slurry under shearing and heating for an amount of time to cause disruption of inter- or intra-polymer linkages of the biomass.

2-3. (canceled)

4. The process of claim 1, wherein the solvent is at least one polyhydric alcohol having from 1 to 6 carbon atoms and having from 1 to 4 hydroxyl groups.

5. (canceled)

6. The process of claim 1, wherein the solvent is at least one polyhydric alcohol chosen from at least one of 1,6-anhydro-glucose, 2,5-anhydro-D-mannitol, 1,2,6-hexanetriol, arabitol, adonitol, butanetriol, dulcitol, diethylene glycol, diglycerol, erythritol, ethanol, ethylene glycol, fucitol, galactol, glycerol, iditol, inositol, isomalt, lacitol, maltitol, maltotetraitol, maltotriitol, mannitol, mesoerythritol, methanol, polyethylene glycol, polyglycitol, polyglycerol, ribitol, scyllitol, sorbitol, triethylene glycol, triglycerol, trimethylolpropane, threitol, volemitol, and xylitol.

7. The process of claim 6, wherein the solvent is glycerol.

8-10. (canceled)

11. The process of claim 1, wherein the mixer is pre-heated to a temperature in the range of 100° C. to 300° C. prior to adding the biomass to the mixer.

12-13. (canceled)

14. The process of claim 1, wherein the lignocellulosic biomass is present relative to the solvent in a biomass:solvent weight ratio of between 1:5 and 5:1.

15-20. (canceled)

21. The process of claim 1, further comprising producing at least one fermentable sugar by hydrolyzing the biomass slurry with at least one enzyme.

22. The process of claim 21, wherein the solvent is at least one polyhydric alcohol chosen from at least one of 1,6-anhydro-glucose, 2,5-anhydro-D-mannitol, 1,2,6-hexanetriol, arabitol, adonitol, butanetriol, dulcitol, diethylene glycol, diglycerol, erythritol, ethanol, ethylene glycol, fucitol, galactol, glycerol, iditol, inositol, isomalt, lacitol, maltitol, maltotetraitol, maltotriitol, mannitol, mesoerythritol, methanol, polyethylene glycol, polyglycitol, polyglycerol, ribitol, scyllitol, sorbitol, triethylene glycol, triglycerol, trimethylolpropane, threitol, volemitol, and xylitol.

23. The process of claim 22, wherein the at least one polyhydric alcohol is glycerol.

24. (canceled)

25. The process of claim 21, wherein the lignocellulosic biomass is present relative to the solvent in a biomass:solvent weight ratio of between 1:5 and 5:1.

26-31. (canceled)

32. The process of claim 1, further comprising fractionating lignin from the biomass slurry in a manner that provides lignin having a number average molar mass (Mn) in the range of 5,000 to 6,000 daltons.

33. The process of claim 32, wherein the solvent is at least one polyhydric alcohol chosen from 1,6-anhydro-glucose, 2,5-anhydro-D-mannitol, 1,2,6-hexanetriol, arabitol, adonitol, butanetriol, dulcitol, diethylene glycol, diglycerol, erythritol, ethanol, ethylene glycol, fucitol, galactol, glycerol, iditol, inositol, isomalt, lacitol, maltitol, maltotetraitol, maltotriitol, mannitol, mesoerythritol, methanol, polyethylene glycol, polyglycitol, polyglycerol, ribitol, scyllitol, sorbitol, triethylene glycol, triglycerol, trimethylolpropane, threitol, volemitol, xylitol, and combinations thereof.

34. The process of claim 33, wherein the at least one polyhydric alcohol is glycerol.

35. (canceled)

36. The process of claim 32, wherein the lignocellulosic biomass is present relative to the solvent in a biomass:solvent weight ratio of between 1:5 and 5:1.

37-42. (canceled)

43. A method of processing lignocellulosic biomass, comprising:
mixing lignocellulosic biomass and glycerol to form a biomass slurry; and
heating and shearing the biomass slurry at a temperature ranging from 100° C. to 300° C. for an amount of time to disrupt inter- or intra-polymer linkages of the biomass.

44. A composition of matter comprising a lignin having a number average molar mass (Mn) in the range of 1,000 to 10,000 daltons wherein the lignin has phenolic hydroxyl content.

45. The composition of matter of claim 44, wherein the lignin is substituted with at least one functional group selected from the group consisting of a syringyl phenolic group, a guaiacyl phenolic group, a p-hydroxyl phenolic group, and combinations thereof.

46. The composition of matter of claim 44, wherein the composition has no sulfur content.

47. The composition of matter of claim 44, wherein the lignin has a Mn of 5,784 daltons.

48. The composition of matter of claim 44, wherein the lignin has a weight average molecular weight (Mw) in the range of 19,000 to 20,000 daltons.

49. (canceled)
